SEQUENCE LISTING (30 sequences)

SEQ ID NO: 1 - protein, 5 aa, Artificial Sequence
  source: /note=“Description of Artificial Sequence Synthetic peptide”
TYTMS

SEQ ID NO: 2 - protein, 18 aa, Artificial Sequence
  source: /note=“Description of Artificial Sequence Synthetic peptide”
TISPGDSFGYYYPDSVQG

SEQ ID NO: 3 - protein, 12 aa, Artificial Sequence
  source: /note=“Description of Artificial Sequence Synthetic peptide”
DIYYNYGAWFAY

SEQ ID NO: 4 - protein, 15 aa, Artificial Sequence
  source: /note=“Description of Artificial Sequence Synthetic peptide”
KASQSVDYDGDSYMN

SEQ ID NO: 5 - protein, 7 aa, Artificial Sequence
  source: /note=“Description of Artificial Sequence Synthetic peptide”
AASTLES

SEQ ID NO: 6 - protein, 9 aa, Artificial Sequence
  source: /note=“Description of Artificial Sequence Synthetic peptide”
QQANEDPRT

SEQ ID NO: 7 - protein, 122 aa, Artificial Sequence
  source: /note=“Description of Artificial Sequence Synthetic polypeptide”
DVQLVESGGGLVKPGGSLRLSCAASGFTFSTYTMSWVRQAPGKGLEWVATISPGDSFGYYYPDSVQGRFTISRDNAKNSLYLQMNSLRAEDTAVYYCTRDIYYNYGAWFAYWGQGTLVTVSS

SEQ ID NO: 8 - protein, 111 aa, Artificial Sequence
  source: /note=“Description of Artificial Sequence Synthetic polypeptide”
DIQLTQSPSSLSASVGDRVTITCKASQSVDYDGDSYMNWYQQKPGKAPKLLIYAASTLESGVPSRFSGSGSGTDFTLTISSLQPEDFATYYCQQANEDPRTFGQGTKVEIK

SEQ ID NO: 9 - protein, 451 aa, Artificial Sequence
  source: /note=“Description of Artificial Sequence Synthetic polypeptide”
DVQLVESGGGLVKPGGSLRLSCAASGFTFSTYTMSWVRQAPGKGLEWVATISPGDSFGYYYPDSVQGRFTISRDNAKNSLYLQMNSLRAEDTAVYYCTRDIYYNYGAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 218 aa, Artificial Sequence
  source: /note=“Description of Artificial Sequence Synthetic polypeptide”
DIQLTQSPSSLSASVGDRVTITCKASQSVDYDGDSYMNWYQQKPGKAPKLLIYAASTLESGVPSRFSGSGSGTDFTLTISSLQPEDFATYYCQQANEDPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 7 aa, Artificial Sequence
  source: /note=“Description of Artificial Sequence Synthetic peptide”
GFTFSTY

SEQ ID NO: 12 - protein, 7 aa, Artificial Sequence
  source: /note=“Description of Artificial Sequence Synthetic peptide”
SPGDSFG

SEQ ID NO: 13 - protein, 12 aa, Artificial Sequence
  source: /note=“Description of Artificial Sequence Synthetic peptide”
DIYYNYGAWFAY

SEQ ID NO: 14 - protein, 15 aa, Artificial Sequence
  source: /note=“Description of Artificial Sequence Synthetic peptide”
KASQSVDYDGDSYMN

SEQ ID NO: 15 - protein, 7 aa, Artificial Sequence
  source: /note=“Description of Artificial Sequence Synthetic peptide”
AASTLES

SEQ ID NO: 16 - protein, 9 aa, Artificial Sequence
  source: /note=“Description of Artificial Sequence Synthetic peptide”
QQANEDPRT

SEQ ID NO: 17 - protein, 10 aa, Artificial Sequence
  source: /note=“Description of Artificial Sequence Synthetic peptide”
GFTFSTYTMS

SEQ ID NO: 18 - protein, 11 aa, Artificial Sequence
  source: /note=“Description of Artificial Sequence Synthetic peptide”
TISPGDSFGYY

SEQ ID NO: 19 - protein, 12 aa, Artificial Sequence
  source: /note=“Description of Artificial Sequence Synthetic peptide”
DIYYNYGAWFAY

SEQ ID NO: 20 - protein, 15 aa, Artificial Sequence
  source: /note=“Description of Artificial Sequence Synthetic peptide”
KASQSVDYDGDSYMN

SEQ ID NO: 21 - protein, 7 aa, Artificial Sequence
  source: /note=“Description of Artificial Sequence Synthetic peptide”
AASTLES

SEQ ID NO: 22 - protein, 9 aa, Artificial Sequence
  source: /note=“Description of Artificial Sequence Synthetic peptide”
QQANEDPRT

SEQ ID NO: 23 - protein, 6 aa, Artificial Sequence
  source: /note=“Description of Artificial Sequence Synthetic peptide”
STYTMS

SEQ ID NO: 24 - protein, 14 aa, Artificial Sequence
  source: /note=“Description of Artificial Sequence Synthetic peptide”
WVATISPGDSFGYY

SEQ ID NO: 25 - protein, 13 aa, Artificial Sequence
  source: /note=“Description of Artificial Sequence Synthetic peptide”
TRDIYYNYGAWFA

SEQ ID NO: 26 - protein, 11 aa, Artificial Sequence
  source: /note=“Description of Artificial Sequence Synthetic peptide”
DYDGDSYMNWY

SEQ ID NO: 27 - protein, 10 aa, Artificial Sequence
  source: /note=“Description of Artificial Sequence Synthetic peptide”
LLIYAASTLE

SEQ ID NO: 28 - protein, 8 aa, Artificial Sequence
  source: /note=“Description of Artificial Sequence Synthetic peptide”
QQANEDPR

SEQ ID NO: 29 - protein, 213 aa, Homo sapiens
MVPEEEPQDREKGLWWFQLKVWSMAVVSILLLSVCFTVSSVVPHNFMYSKTVKRLSKLREYQQYHPSLTCVMEGKDIEDWSCCPTPWTSFQSSCYFISTGMQSWTKSQKNCSVMGADLVVINTREEQDFIIQNLKRNSSYFLGLSDPGGRRHWQWVDQTPYNENVTFWHSGEPNNLDERCAIINFRSSEEWGWNDIHCHVPQKSICKMKKIYI

SEQ ID NO: 30 - protein, 86 aa, Homo sapiens
MIPAVVLLLLLLVEQAAALGEPQLCYILDAILFLYGIVLTLLYCRLKIQVRKAAITSYEKSDGVYTGLSTRNQETYETLKHEKPPQ

(19) United States
(12) Patent Application Publication
(10) Pub. No.: US 2024/0018246 A1
(43) Pub. Date: Jan. 18, 2024

(54) METHODS OF TREATING CUTANEOUS LUPUS ERYTHEMATOSUS AND SYSTEMIC LUPUS ERYTHEMATOSUS

(71) Applicant: Biogen MA Inc., Cambridge, MA (US)

(72) Inventors: Francois Gaudreault, Littleton, MA (US); Himanshu Naik, Winchester, MA (US)

(21) Appl. No.: 18/038,911

(22) PCT Filed: Dec. 3, 2021
(86) PCT No.: PCT/US2021/061764

§ 371 Date: May 25, 2023

Related U.S. Application Data
(60) Provisional application No. 63/121,194, filed on Dec. 3, 2020.

(51) Int. Cl. C07K 16/28; A61K 47/26; A61K 47/22; A61K 47/18; A61K 47/42; A61K 9/00; A61P 37/06

(52) U.S. Cl. CPC C07K 16/2851; A61K 47/26; A61K 47/22; A61K 47/183; A61K 47/42; A61K 9/0019; A61P 37/06; C07K 2317/24; A61K 2039/505

(57) ABSTRACT

Dosage regimens of anti-Blood Dendritic Cell Antigen 2 antibodies are provided for use in the treatment of cutaneous lupus erythematosus and systemic lupus erythematosus.

### CROSS-REFERENCE TO RELATED APPLICATIONS

This application claims the benefit of priority of U.S. Provisional Appl. No. 63/121,194 filed Dec. 3, 2020, the contents of which are incorporated by reference herein in their entirety.

### SEQUENCE LISTING

The instant application contains a Sequence Listing which has been submitted electronically in ASCII format and is hereby incorporated by reference in its entirety. Said ASCII copy, created on Nov. 12, 2021, is named 13751-0337WO1_SL.txt and is 17,573 bytes in size.

### FIELD OF THE INVENTION

The present application relates to the clinical use of anti-Blood Dendritic Cell Antigen 2 antibodies in the treatment of cutaneous lupus erythematosus and systemic lupus erythematosus.

### BACKGROUND

Blood dendritic cell antigen 2 (BDCA2) is a C-type lectin expressed on human plasmacytoid dendritic cells (pDCs) (Dzionek et al., J. Immunol., 165:6037-6046 (2000)), a specialized population of bone marrow-derived cells that secrete type I interferons (IFNs) in response to toll-like receptor (TLR) ligands. BDCA2 consists of a single extracellular carbohydrate recognition domain (CRD), which belongs to the type II C-type lectin group, at its C-terminus, a transmembrane region, and a short cytoplasmic tail at its N-terminus that does not harbor a signaling motif. BDCA2 transmits intracellular signals through an associated transmembrane adaptor, the FcεRIγ, and induces a B cell receptor (BCR)-like signaling cascade.

Cutaneous lupus erythematosus (CLE) is an autoimmune disease that impacts the skin and may present with or without systemic manifestations.

Systemic lupus erythematosus (SLE) is a chronic, complex autoimmune disease that affects multiple organ systems and is unpredictable in disease severity, with periods of illness or flares alternating with periods of remission.

Given the lack of specific treatments for CLE and SLE and the high impact of the disease on quality of life, a large unmet need remains to develop new targeted and efficacious therapies.

### SUMMARY

This disclosure relates, in part, to dosage regimens of anti-BDCA2 antibodies for use in the treatment of CLE and SLE.

In a first aspect, the disclosure features a method of treating CLE or SLE in a human subject in need thereof. The method comprises administering subcutaneously to the human subject an anti-BDCA2 antibody at a dose of 225 mg every four weeks. The anti-BDCA2 antibody comprises an immunoglobulin heavy chain variable domain (VH) and an immunoglobulin light chain variable domain (VL), the VH and VL, respectively, comprising: VH complementarity determining regions (CDRs) VH-CDR1, VH-CDR2, and VH-CDR3, wherein VH-CDR1 consists of the amino acid sequence set forth in SEQ ID NO:1; VH-CDR2 consists of the amino acid sequence set forth in SEQ ID NO:2; and VH-CDR3 consists of the amino acid sequence set forth in SEQ ID NO:3; and VL CDRs VL-CDR1, VL-CDR2, and VL-CDR3, wherein VL-CDR1 consists of the amino acid sequence set forth in SEQ ID NO:4; VL-CDR2 consists of the amino acid sequence set forth in SEQ ID NO:5; and VL-CDR3 consists of the amino acid sequence set forth in SEQ ID NO:6. In some instances, the human subject is administered a loading dose of the anti-BDCA2 antibody two weeks after the first administration of the anti-BDCA2 antibody. In some cases, the loading dose is 225 mg. In some cases, the loading dose is 450 mg. The patient population can be adult or pediatric CLE or adult or pediatric SLE.

The disclosure also features a method of treating lupus nephritis, neuropsychiatric lupus (NPSLE), Sjogren syndrome, systemic sclerosis (scleroderma), morphea, psoriasis, rheumatoid arthritis, inflammatory bowel disease (IBD), dermatomyositis, polymyositis, type I diabetes, or cytokine release syndrome in a human subject in need thereof. The patient population for any of these indications can be adult or pediatric. The method comprises administering subcutaneously to the human subject an anti-BDCA2 antibody at a dose of 225 mg every four weeks. The anti-BDCA2 antibody comprises an immunoglobulin heavy chain variable domain (VH) and an immunoglobulin light chain variable domain (VL), the VH and VL, respectively, comprising: VH complementarity determining regions (CDRs) VH-CDR1, VH-CDR2, and VH-CDR3, wherein VH-CDR1 consists of the amino acid sequence set forth in SEQ ID NO:1; VH-CDR2 consists of the amino acid sequence set forth in SEQ ID NO:2; and VH-CDR3 consists of the amino acid sequence set forth in SEQ ID NO:3; and VL CDRs VL-CDR1, VL-CDR2, and VL-CDR3, wherein VL-CDR1 consists of the amino acid sequence set forth in SEQ ID NO:4; VL-CDR2 consists of the amino acid sequence set forth in SEQ ID NO:5; and VL-CDR3 consists of the amino acid sequence set forth in SEQ ID NO:6. In some instances, the human subject is administered a loading dose of the anti-BDCA2 antibody two weeks after the first administration of the anti-BDCA2 antibody. In some cases, the loading dose is 225 mg. In some cases, the loading dose is 450 mg.

In certain instances, the human subject is administered a second loading dose of the anti-BDCA2 antibody 12, 13, 14, 15, 16, 17, 18, 19, 20, 21, 22, 23, 24, 25, or 26 weeks (e.g., eighteen weeks) after the first administration of the anti-BDCA2 antibody. In some cases, the second loading dose is 225 mg. In some cases, the second loading dose is 450 mg.

In some instances, the anti-BDCA2 antibody is administered at a dose of 225 mg every 21, 22, 23, 24, 25, 26, 27, 28, 29, 30, 31, 32, 33, 34, or 35 days (e.g., every four weeks) over at least 16 weeks. In some instances, the anti-BDCA2 antibody is administered at a dose of 225 mg once a month over at least 16 weeks. In some cases, the anti-BDCA2 antibody is administered at a dose of 225 mg every four weeks over at least 52 weeks. In some cases, the anti-BDCA2 antibody is administered indefinitely at a dose of 225 mg every four weeks. In some cases, the anti-BDCA2 antibody is administered at a dose of 225 mg every four weeks until the health care practitioner deems it is no longer necessary. In some cases, the anti-BDCA2 antibody is administered at a dose of 225 mg every four weeks over the lifetime of the patient (i.e., a chronic use).

In certain instances, at least four doses (e.g., 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 41, 42, 43, 44, 45, 46, 47, 48, 49, 50, 51, 52, etc.) of the anti-BDCA2 antibody are administered to the human subject. In some cases, at least twelve doses of the anti-BDCA2 antibody are administered to the human subject. In other cases, at least thirteen doses of the anti-BDCA2 antibody are administered to the human subject. In certain cases, at least fourteen doses of the anti-BDCA2 antibody are administered to the human subject. In some cases, at least fifteen doses of the anti-BDCA2 antibody are administered to the human subject. In other cases, at least sixteen doses of the anti-BDCA2 antibody are administered to the human subject. In other cases, doses of the anti-BDCA2 antibody are administered to the human subject until the health care practitioner deems it is no longer necessary. In other cases, at least sixteen doses of the anti-BDCA2 antibody are administered to the human subject. In other cases, doses of the anti-BDCA2 antibody are administered to the human subject over the lifetime of the patient (i.e., a chronic use).

In some instances, the CLE disease is mild CLE activity. In some instances, the CLE disease is moderate CLE activity. In other instances, the CLE disease is severe CLE activity. In some cases, the CLE type is acute CLE (ACLE). In some cases, the CLE type is subacute CLE (SCLE). In some cases, the CLE type is chronic CLE (CCLE). In certain cases, the CLE is discoid lupus erythematosus (DLE). In some cases, the CLE is active CLE. In some cases, the CLE is active CLE and the human subject is intolerant and/or refractory to antimalarial and topical steroid therapy. In certain cases, the active CLE is CLE with systemic manifestations of lupus and the human subject is intolerant and/or refractory to antimalarial and topical steroid therapy. In some cases, the active CLE is CLE without systemic manifestations of lupus and the human subject is intolerant or refractory to antimalarial and/or topical steroid therapy. In certain instances, the human subject achieves clinically meaningful reduction, e.g., a 4-point decrease, in Cutaneous Lupus Erythematosus Disease Area and Severity Index-A (CLASI-A) score from baseline about sixteen weeks to about 24 weeks after the first administration of the anti-BDCA2 antibody. In some cases, the human subject achieves clinically meaningful reduction from baseline in disease activity on an Investigator Global assessment (IGA) specific for CLE (CLA-IGA-R), e.g., a score of 0, 1, 2, or 3, about sixteen weeks to about 24 weeks after the first administration of the anti-BDCA2 antibody.

In certain instances, the SLE is active SLE. In some cases, the human subject has active, autoantibody-positive SLE. In certain cases, wherein the human subject has active, autoantibody-positive SLE and the human subject is receiving the non-biologic standard of care therapy for SLE. In some cases, the SLE is moderate SLE. In certain cases, the SLE is severe SLE. In some cases, the SLE is active SLE with active joint and/or skin manifestations. In some cases, the human subject has a SLEDAI-2K≥6 excluding alopecia, lupus-related headache and organic brain disease at initiation of treatment. In certain cases, the human subject has a clinical SLEDAI-2K≥4 excluding alopecia, lupus-related headache and organic brain disease, anti-ds DNA, low complement C3 and/or C4, or fever, at initiation of treatment. In certain cases, the human subject has BILAG-2004 grade A in ≥1 organ system or BILAG-2004 grade B in ≥2 organ systems at initiation of treatment. In certain instances, the human subject achieves an SRI-4 response about 3, 4, 5, 6, 7, 8, 9, 10, 12, 16, 24, or 52 weeks after initiation of treatment with the anti-BDCA2 antibody. In certain instances, the human subject achieves a Joint-50 response rate about 3, 4, 5, 6, 7, 8, 9, 10, 12, 14, 16, 24, or 52 weeks after initiation of treatment with the anti-BDCA2 antibody in a human subject who had at least 4 joints that are swollen and tender at initiation of treatment.

In some instances, the anti-BDCA2 antibody is formulated as a sterile, liquid pharmaceutical composition comprising the anti-BDCA2 antibody at a concentration of 150 mg/ml; sucrose at a concentration of 3%; L-histidine at a concentration of 20 mM; L-Arginine HCl at a concentration of 100 mM; glutathione (GSH or a combination of GSH and GSSG) at concentration of 0.4 mM; and polysorbate 80 (PS80) at a concentration of wherein the pharmaceutical composition has a pH of 5.7.

In some instances, the VH comprises or consists of a sequence at least 80% identical to the amino acid sequence of SEQ ID NO:7 and the VL comprises or consists of a sequence at least 80% identical to the amino acid sequence of SEQ ID NO:8. In certain instances, the VH comprises or consists of a sequence at least 90% identical to the amino acid sequence of SEQ ID NO:7 and the VL comprises or consists of a sequence at least 90% identical to the amino acid sequence of SEQ ID NO:8. In other instances, the VH comprises or consists of the amino acid sequence set forth in SEQ ID NO:7 and the VL comprises or consists of the amino acid sequence set forth in SEQ ID NO:8.

In some instances, the anti-BDCA2 antibody used in the methods described herein comprises an immunoglobulin heavy chain variable domain (VH) and an immunoglobulin light chain variable domain (VL), the VH and VL, respectively, comprising: VH complementarity determining regions (CDRs) VH-CDR1, VH-CDR2, and VH-CDR3, wherein VH-CDR1 comprises or consists of the amino acid sequence set forth in SEQ ID NO:1; VH-CDR2 comprises or consists of the amino acid sequence set forth in SEQ ID NO:2; and VH-CDR3 comprises or consists of the amino acid sequence set forth in SEQ ID NO:3; and VL CDRs VL-CDR1, VL-CDR2, and VL-CDR3, wherein VL-CDR1 comprises or consists of the amino acid sequence set forth in SEQ ID NO:4; VL-CDR2 comprises or consists of the amino acid sequence set forth in SEQ ID NO:5; and VL-CDR3 comprises or consists of the amino acid sequence set forth in SEQ ID NO:6, wherein the VH comprises or consists of a sequence at least 80% identical to the amino acid sequence of SEQ ID NO:7 and the VL comprises or consists of a sequence at least 80% identical to the amino acid sequence of SEQ ID NO:8.

In some instances, the anti-BDCA2 antibody used in the methods described herein comprises an immunoglobulin heavy chain variable domain (VH) and an immunoglobulin light chain variable domain (VL), the VH and VL, respectively, comprising: VH complementarity determining regions (CDRs) VH-CDR1, VH-CDR2, and VH-CDR3, wherein VH-CDR1 comprises or consists of the amino acid sequence set forth in SEQ ID NO:1; VH-CDR2 comprises or consists of the amino acid sequence set forth in SEQ ID NO:2; and VH-CDR3 comprises or consists of the amino acid sequence set forth in SEQ ID NO:3; and VL CDRs VL-CDR1, VL-CDR2, and VL-CDR3, wherein VL-CDR1 comprises or consists of the amino acid sequence set forth in SEQ ID NO:4; VL-CDR2 comprises or consists of the amino acid sequence set forth in SEQ ID NO:5; and VL-CDR3 comprises or consists of the amino acid sequence set forth in SEQ ID NO:6, wherein the VH comprises or consists of a sequence at least 90% identical to the amino acid sequence of SEQ ID NO:7 and the VL comprises or consists of a sequence at least 90% identical to the amino acid sequence of SEQ ID NO:8.

In some instances, the anti-BDCA2 antibody used in the methods described herein comprises an immunoglobulin heavy chain variable domain (VH) and an immunoglobulin light chain variable domain (VL), the VH and VL, respectively, comprising: VH complementarity determining regions (CDRs) VH-CDR1, VH-CDR2, and VH-CDR3, wherein VH-CDR1 comprises or consists of the amino acid sequence set forth in SEQ ID NO:1; VH-CDR2 comprises or consists of the amino acid sequence set forth in SEQ ID NO:2; and VH-CDR3 comprises or consists of the amino acid sequence set forth in SEQ ID NO:3; and VL CDRs VL-CDR1, VL-CDR2, and VL-CDR3, wherein VL-CDR1 comprises or consists of the amino acid sequence set forth in SEQ ID NO:4; VL-CDR2 comprises or consists of the amino acid sequence set forth in SEQ ID NO:5; and VL-CDR3 comprises or consists of the amino acid sequence set forth in SEQ ID NO:6, wherein the VH comprises or consists of a sequence at least 95% identical to the amino acid sequence of SEQ ID NO:7 and the VL comprises or consists of a sequence at least 95% identical to the amino acid sequence of SEQ ID NO:8.

In some instances, the anti-BDCA2 antibody used in the methods described herein comprises an immunoglobulin heavy chain variable domain (VH) and an immunoglobulin light chain variable domain (VL), the VH and VL, respectively, comprising: VH complementarity determining regions (CDRs) VH-CDR1, VH-CDR2, and VH-CDR3, wherein VH-CDR1 comprises or consists of the amino acid sequence set forth in SEQ ID NO:1; VH-CDR2 comprises or consists of the amino acid sequence set forth in SEQ ID NO:2; and VH-CDR3 comprises or consists of the amino acid sequence set forth in SEQ ID NO:3; and VL CDRs VL-CDR1, VL-CDR2, and VL-CDR3, wherein VL-CDR1 comprises or consists of the amino acid sequence set forth in SEQ ID NO:4; VL-CDR2 comprises or consists of the amino acid sequence set forth in SEQ ID NO:5; and VL-CDR3 comprises or consists of the amino acid sequence set forth in SEQ ID NO:6, wherein the VH comprises or consists of a sequence at least 97% identical to the amino acid sequence of SEQ ID NO:7 and the VL comprises or consists of a sequence at least 97% identical to the amino acid sequence of SEQ ID NO:8.

In some instances, the anti-BDCA2 antibody used in the methods described herein comprises an immunoglobulin heavy chain variable domain (VH) and an immunoglobulin light chain variable domain (VL), the VH and VL, respectively, comprising: VH complementarity determining regions (CDRs) VH-CDR1, VH-CDR2, and VH-CDR3, wherein VH-CDR1 comprises or consists of the amino acid sequence set forth in SEQ ID NO:1; VH-CDR2 comprises or consists of the amino acid sequence set forth in SEQ ID NO:2; and VH-CDR3 comprises or consists of the amino acid sequence set forth in SEQ ID NO:3; and VL CDRs VL-CDR1, VL-CDR2, and VL-CDR3, wherein VL-CDR1 comprises or consists of the amino acid sequence set forth in SEQ ID NO:4; VL-CDR2 comprises or consists of the amino acid sequence set forth in SEQ ID NO:5; and VL-CDR3 comprises or consists of the amino acid sequence set forth in SEQ ID NO:6, wherein the VH comprises or consists of a sequence at least 99% identical to the amino acid sequence of SEQ ID NO:7 and the VL comprises or consists of a sequence at least 99% identical to the amino acid sequence of SEQ ID NO:8.

In some instances, the anti-BDCA2 antibody used in the methods described herein is an antibody described in U.S. Pat. No. 9,902,775, which is incorporated herein by reference.

In certain instances, the anti-BDCA2 antibody comprises an immunoglobulin heavy chain and an immunoglobulin light chain. In some cases, the heavy chain comprises or consists of a sequence at least 80% identical to the amino acid sequence of SEQ ID NO:9 and the light chain comprises or consists of a sequence at least 80% identical to the amino acid sequence of SEQ ID NO:10. In other cases, the heavy chain comprises or consists of a sequence at least 90% identical to the amino acid sequence of SEQ ID NO:9 and the light chain comprises or consists of a sequence at least 90% identical to the amino acid sequence of SEQ ID NO:10. In some cases, the heavy chain comprises or consists of the amino acid sequence set forth in SEQ ID NO:9 and the light chain comprises or consists of the amino acid sequence set forth in SEQ ID NO:10.

In some instances, the method further involves administering to the human subject at least one of an antimalarial, a corticosteroid, an immunosuppressive drug, or an anti-B-lymphocyte stimulator (BLyS) monoclonal antibody. In certain instances, the method further involves administering to the human subject at least one of a mycophenolate, an azathioprine, methotrexate, a calcineurin inhibitor, or cyclophosphamide.

In another aspect the disclosure features a pre-filled syringe comprising a sterile preparation of an anti-BDCA2 antibody. The pre-filled syringe is adapted for subcutaneous administration of the anti-BDCA2 antibody at a fixed dose of 225 mg. The anti-BDCA2 antibody comprises an immunoglobulin heavy chain variable domain (VH) and an immunoglobulin light chain variable domain (VL), the VH and VL, respectively, comprising VH complementarity determining regions (CDRs) VH-CDR1, VH-CDR2, and VH-CDR3, wherein VH-CDR1 comprises or consists of the amino acid sequence set forth in SEQ ID NO:1; VH-CDR2 comprises or consists of the amino acid sequence set forth in SEQ ID NO:2; and VH-CDR3 comprises or consists of the amino acid sequence set forth in SEQ ID NO:3; and VL CDRs VL-CDR1, VL-CDR2, and VL-CDR3, wherein VL-CDR1 comprises or consists of the amino acid sequence set forth in SEQ ID NO:4; VL-CDR2 comprises or consists of the amino acid sequence set forth in SEQ ID NO:5; and VL-CDR3 comprises or consists of the amino acid sequence set forth in SEQ ID NO:6.

A pre-filled syringe described herein may be assembled with device components such as a finger-flange and a safety needle shield to facilitate administration. It also may be assembled in an auto-injector to facilitate self-administration by patients and/or administration by care givers.

In some instances, the VH comprises or consists of a sequence at least 80% identical to the amino acid sequence of SEQ ID NO:7 and the VL comprises or consists of a sequence at least 80% identical to the amino acid sequence of SEQ ID NO:8. In certain instances, the VH comprises or consists of a sequence at least 90% identical to the amino acid sequence of SEQ ID NO:7 and the VL comprises or consists of a sequence at least 90% identical to the amino acid sequence of SEQ ID NO:8. In other instances, the VH comprises or consists of the amino acid sequence set forth in SEQ ID NO:7 and the VL comprises or consists of the amino acid sequence set forth in SEQ ID NO:8.

In other instances, the anti-BDCA2 antibody comprises an immunoglobulin heavy chain and an immunoglobulin light chain. In some cases, the heavy chain comprises or consists of a sequence at least 80% identical to the amino acid sequence of SEQ ID NO:9 and the light chain comprises or consists of a sequence at least 80% identical to the amino acid sequence of SEQ ID NO:10. In other cases, the heavy chain comprises or consists of a sequence at least 90% identical to the amino acid sequence of SEQ ID NO:9 and the light chain comprises or consists of a sequence at least 90% identical to the amino acid sequence of SEQ ID NO:10. In some cases, the heavy chain comprises or consists of the amino acid sequence set forth in SEQ ID NO:9 and the light chain comprises or consists of the amino acid sequence set forth in SEQ ID NO:10.

In some instances, the anti-BDCA2 antibody is formulated as a sterile, liquid pharmaceutical composition comprising the anti-BDCA2 antibody at a concentration of 150 mg/ml; sucrose at a concentration of 3%; L-histidine at a concentration of 20 mM; L-Arginine HCl at a concentration of 100 mM; glutathione (GSH or a combination of GSH and GSSG) at a concentration of 0.4 mM; and polysorbate 80 (PS80) at a concentration of 0.05%, wherein the pharmaceutical composition has a pH of 5.7.

In some instances, the syringe is a United States Pharmacopeia or European Pharmacopeia, Type 1, clear glass syringe that is stoppered with a rubber stopper.

In some instances, the syringe is 2.25 mL pre-fillable syringe with Type I glass and with a butyl rubber plunger (ethylene tetrafluoroethylene-coated).

For the avoidance of any doubt it is emphasized that the expressions “in some embodiments”, “in a certain embodiments”, “in certain instances”, “in some instances”, “in a further embodiment”, “in one embodiment” and “in a further embodiment” and the like are used and meant such that any of the embodiments described therein are to be read with a mind to combine each of the features of those embodiments and that the disclosure has to be treated in the same way as if the combination of the features of those embodiments would be spelled out in one embodiment. The same is true for any combination of embodiments and features of the appended claims and illustrated in the Examples, which are also intended to be combined with features from corresponding embodiments disclosed in the description, wherein only for the sake of consistency and conciseness the embodiments are characterized by dependencies while in fact each embodiment and combination of features, which could be construed due to the (multiple) dependencies must be seen to be literally disclosed and not considered as a selection among different choices. In this context, the person skilled in the art will appreciate that the embodiments and features disclosed in the Examples are intended to be generalized to equivalents having the same function as those exemplified therein.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description and from the claims.

### BRIEF DESCRIPTION OF THE DRAWINGS

FIG. 1 depicts the BIIB059 pharmacokinetic time-course across treatments in the Phase 2 study. Observed median concentrations (dots) at each BIIB059 dose of 50 mg (bottom most curve), 150 mg (middle curve) and 450 mg (top curve) against the corresponding 90% prediction interval (shaded area) (n=500) in participants with CLE from the Phase 2 study. The thick lines are the median projections. BDCA2 target engagement level (0.64 μg/mL), the IFN-α IC90 levels (9.7 μg/mL), and the 3×IC90 level are shown.

FIG. 2 provides Forest Plots of CLASI-A Score: Percent Change from Baseline at Week 16, Mixed Model for Repeated Measures Subgroup Analysis.

FIG. 3A-3B provides exposure-response analysis from the Phase 2 study. FIG. 3A depicts the observed versus simulated for the exposure—response model using trough concentration at week 16 for CLASI-A in the Phase 2 study. The observed change from baseline in CLASI-A and the associated standard deviations were determined according to 4 bins of the observed BIIB059 trough concentrations (plus readouts when receiving placebo, i.e. concentration being 0) and were plotted at the median exposure within each bin. The solid lines are the simulated median exposure—response trend responses (1,000 replicates). The shaded areas indicate the associated 90% CI. FIG. 3B are boxplots showing the range of observed trough concentrations (median, interquartile and 5th/95th percentile). EC90 is the BDAC2 target engagement of 0.64 μg/mL. IFN90 is the inhibitory level of 9.7 μg/mL.

FIG. 4 is a graph showing the fraction of participants with BIIB059 levels greater or equal than the CLASI-A EC90 (10.1 μg/mL) at week 16. Simulation based on 1,000 Phase 3 trials at BIIB059 doses ranging from 50 mg to 450 mg Q4W with a loading dose at week 2 (loading dose in same amount as first dose administered), assuming an ADA incidence rate of 19%. The dash line represents 80% of the participants.

FIG. 5 shows a pharmacokinetic time-course at the 150 mg and 225 mg doses of BIIB059. BIIB059 PK time-course simulated (n=1,000) after 150 mg (left) and 225 mg (right) administered Q4W in patients with CLE. Observed PK timepoints are shown by filled in circles (ADA positive) and circles (ADA negative) at the 150 mg dose in the Phase 2 study. The 80% prediction interval is shown in the shaded area, along with the median by a solid black line. The dash blue lines depict the estimated EC90 for CLASI-A (10.1 μg/mL), where are the IFNα IC90 (9.7 μg/mL) is shown in black. For the 225 mg (right), an ADA incidence rate of 19% was assumed.

FIG. 6A is a schematic diagram of the Phase 3 CLE study design.

FIG. 6B is an alternate schematic diagram of a CLE study design.

FIG. 7 is a BIIB059 pharmacokinetic time course graph across treatments in participants with SLE. Observed median concentrations (dots) at each BIIB059 dose of 50 mg (bottom trace), 150 mg (middle trace) and 450 mg (top trace) against the corresponding 90% prediction interval (n=500) in participant with SLE. The thick lines are the median projections for 50 mg, 150 mg, and 450 mg, respectively. BDCA2 target engagement level (0.64 μg/mL) is shown in dashed form towards the bottom. The other dashed line depicts INF-α IC90 levels (9.7 μg/mL), whereas the thick black line from top represent 3×IC90 level. The thick line at top represents the average BIIB059 concentration from the top IV dose of 20 mg/kg considered to be safe and well tolerated in Phase 1.

FIG. 8 is a visual predictive check plot versus BIIB059 Cavg for the Exposure Response SRI-4 Model in participants with SLE. SRI-4 response=reduction in systemic lupus erythematosus response index of ≥4.

FIG. 9 is a depiction of percentage of simulated Phase 3 trials achieving ≥0.17, ≥0.2, ≥0.23, and ≥0.25 Mean Difference in SRI-4 Response relative to placebo based on the Exposure Response Model of SRI-4 at Week 52. SRI-4 response=reduction in systemic lupus erythematosus response index of ≥4.

FIG. 10 shows the pharmacokinetic time course projected in participants with SLE after 225 mg and 450 mg Q4W SC in Phase 3. BIIB059 pharmacokinetic time-course simulated (n=500) after 225 mg and 450 mg administered Q4W SC in patients with SLE. The 80% prediction interval is shown in bottom (225 mg) and top (450 mg) shaded area, along with the medians in black. The top dashed black line depicts the estimated EC90 for CLASI-A (10.1 μg/mL), whereas the IFNa IC90 (9.7 μg/mL) is shown as the bottom dashed line. The top solid line represents the average BIIB059 concentration from the top IV dose of 20 mg/kg considered to be safe and well tolerated in Phase 1. An ADA incidence rate of 19% was assumed.

FIG. 11A is a schematic diagram of the Phase 3 SLE study design.

FIG. 11B is an alternate schematic diagram of a SLE study design.

### DETAILED DESCRIPTION

This application provides dosage regimens of anti-BDCA2 antibodies for use in the treatment of CLE or SLE.

### BDCA2

BDCA2 is a type II C-type lectin that is specifically expressed on plasmacytoid dendritic cells (pDCs). BDCA2 consists of a single extracellular carbohydrate recognition domain (CRD) at its C-terminus, a transmembrane region, and a short cytoplasmic tail at its N-terminus that does not harbor a signaling motif. BDCA2 transmits intracellular signals through an associated transmembrane adaptor, FccRIy. Antibody-mediated ligation of BDCA2 leads to recruitment of spleen tyrosine kinase (SYK) to phosphorylated immunoreceptor tyrosine-based activation motif (ITAM) of FeεRIγ. Syk activation leads to the activation of B cell linker (Blnk), Bruton's tyrosine kinase (BTK), and phospholipase Cγ2 (PLCγ2), leading to Ca2⁺ mobilization.

The amino acid sequence of the human BDCA2 protein (Genbank® Accession No. NP_569708.1) is shown below (the transmembrane domain is italicized; the ectodomain is underlined).

| (SEQ ID NO: 29) | |
|---|---|
| 1 | MVPEEEPQDR EKGLWWFQLK VWSMAVVSIL LLSVCFTVSS VVPHNFMYSK |
| 51 | TVKRLSKLRE YQQYHPSLTC VMEGKDIEDW SCCPTPWTSF QSSCYFISTG |
| 101 | MQSWTKSQKN CSVMGADLVV INTREEQDFI IQNLKRNSSY FLGLSDPGGR |
| 151 | RHWQWVDQTP YNENVTFWHS GEPNNLDERC AIINFRSSEE WGWNDIHCHV |
| 201 | PQKSICKMKK IYI* |

The amino acid sequence of the human FcεRIγ (Genbank® Accession No. NP_004097.1) is shown below.

| (SEQ ID NO: 30) | |
|---|---|
| 1 | MIPAVVLLLL LLVEQAAALG EPQLCYILDA ILFLYGIVLT LLYCRLKIQV |
| 51 | RKAAITSYEK SDGVYTGLST RNQETYETLK HEKPPQ* |

### Anti-BDCA2 Antibodies

In some embodiments, the anti-BDCA2 antibody or BDCA2-binding fragment thereof used in the compositions and methods described herein comprises the three heavy chain variable domain complementarity determining regions (CDRs) of an antibody referred to as “BIIB059.” In some embodiments, the anti-BDCA2 antibody or BDCA2-binding fragment thereof comprises the three light chain variable domain CDRs of BIIB059. In still other embodiments, the anti-BDCA2 antibody or BDCA2-binding fragment thereof comprises the three heavy chain variable domain CDRs and the three light chain variable domain CDRs of BIIB059. The CDRs can be based on any CDR definition in the art, e.g., the definitions of Kabat, Chothia, Chothia from Abysis, enhanced Chothia/AbM, or based on the contact definition. CDR sequences of BIIB059 according to these exemplary CDR definitions are provided in Table A below.

TABLE A
Sequences of the CDRs of BIIB059

| | | Chothia, | Enhanced | |
|---|---|---|---|---|
| Domain | Kabat | from Abysis | Chothia/AbM | Contact |
| VH CDR | TYTMS | GFTFSTY | GFTFSTYTMS | STYTMS |
| | (SEQ ID NO: 1) | (SEQ ID NO: 11) | (SEQ ID NO: 17) | (SEQ ID NO: 23) |
| VH CDR | TISPGDSFGYYYPDSVQG | SPGDSFG | TISPGDSFGYY | WVATISPGDSFGYY |
| | (SEQ ID NO: 2) | (SEQ ID NO: 12) | (SEQ ID NO: 18) | (SEQ ID NO: 24) |
| VH CDR | DIYYNYGAWFAY | DIYYNYGAWFAY | DIYYNYGAWFAY | TRDIYYNYGAWFA |
| | (SEQ ID NO: 3) | (SEQ ID NO: 13) | (SEQ ID NO: 19) | (SEQ ID NO: 25) |
| VL CDR | KASQSVDYDGDSYMN | KASQSVDYDGDSYMN | KASQSVDYDGDSYMN | DYDGDSYMNWY |
| | (SEQ ID NO: 4) | (SEQ ID NO: 14) | (SEQ ID NO: 20) | (SEQ ID NO: 26) |
| VL CDR | AASTLES | AASTLES | AASTLES | LLIYAASTLE |
| | (SEQ ID NO: 5) | (SEQ ID NO: 15) | (SEQ ID NO: 21) | (SEQ ID NO: 27) |
| VL CDR | QQANEDPRT | QQANEDPRT | QQANEDPRT | QQANEDPR |
| | (SEQ ID NO: 6) | (SEQ ID NO: 16) | (SEQ ID NO: 22) | (SEQ ID NO: 28) |

In some embodiments, the anti-BDCA2 antibody or BDCA2-binding fragment thereof comprises a VH CDR1 comprising or consisting of the amino acid sequence set forth in SEQ ID NO.:1 or 17, a VH CDR2 comprising or consisting of the amino acid sequence set forth in SEQ ID NO.: 2; and a VH CDR3 comprising or consisting of the amino acid sequence set forth in SEQ ID NO. 3. In some embodiments, the anti-BDCA2 antibody or BDCA2-binding fragment thereof comprises a VL CDR1 comprising or consisting of the amino acid sequence set forth in SEQ ID NO.:4, a VL CDR2 comprising or consisting of the amino acid sequence set forth in SEQ ID NO.: 5; and a VL CDR3 comprising or consisting of the amino acid sequence set forth in SEQ ID NO. 6.

In certain embodiments, the anti-BDCA2 antibody or BDCA2-binding fragment thereof comprises the CDRs comprising or consisting of the amino acid sequences set forth in SEQ ID NOs.: 1 to 6. In other embodiments, the anti-BDCA2 antibody or BDCA2-binding fragment thereof comprises the CDRs comprising or consisting of the amino acid sequences set forth in SEQ ID NOs.: 11 to 16. In yet other embodiments, the anti-BDCA2 antibody or BDCA2-binding fragment thereof comprises the CDRs comprising or consisting of the amino acid sequences set forth in SEQ ID NOs.: 17 to 22. In yet another embodiment, the anti-BDCA2 antibody or BDCA2-binding fragment thereof comprises the CDRs comprising or consisting of the amino acid sequences set forth in SEQ ID NOs.: 23 to 28. In one embodiment, the anti-BDCA2 antibody or BDCA2-binding fragment thereof comprises a VH CDR1 comprising or consisting of the amino acid sequence set forth in SEQ ID NO.:1 or 17, a VH CDR2 comprising or consisting of the amino acid sequence set forth in SEQ ID NO.: 2; and a VH CDR3 comprising or consisting of the amino acid sequence set forth in SEQ ID NO. 3; and a VL CDR1 comprising or consisting of the amino acid sequence set forth in SEQ ID NO.:4, a VL CDR2 comprising or consisting of the amino acid sequence set forth in SEQ ID NO.: and a VL CDR3 comprising or consisting of the amino acid sequence set forth in SEQ ID NO. 6.

BIIB059 is an exemplary anti-BDCA2 antibody that can be used in the compositions and methods described herein. BIIB059 is a humanized antibody having two glycosylated human IgG1 heavy chains and two human kappa light chains that specifically binds to BDCA2 on the surface of plasmacytoid dendritic cells. The wild-type IgG1 sequence contains a single N-linked glycosylation site and binds to Fc receptors with affinities typical of this class of molecules. BIIB059 is described in U.S. Pat. No. 9,902,775.

The variable heavy chain (VH) of BIIB059 comprises or consists of the following amino acid sequence:

(SEQ ID NO: 7)
DVQLVESGGG LVKPGGSLRL SCAAS [symbol] TYTMSWVRQA
PGKGLEWVAT ISPGDSFGYY YPDSVQGRFT ISRDNAKNSL
YLQMNSLRAE DTAVYYCTRD IYYNYGAWFA YWGQGTLVTV
SS

The variable light chain (VL) of BIIB059 comprises or consists of the following amino acid sequence:

(SEQ ID NO: 8)
DIQLTQSPSS LSASVGDRVT ITCKASQSVD YDGDSYMNWY
QQKPGKAPKL LIYAASTLES GVPSRFSGSG SGTDFTLTIS
SLQPEDFATY YCQQANEDPR TFGQGTKVEI K

In certain embodiments, the anti-BDCA2 antibody or BDCA2-binding fragment thereof comprises a VH having the amino acid sequence set forth in SEQ ID NO:7. In some embodiments, the anti-BDCA2 antibody or antigen-binding fragment thereof selectively binds to the ectodomain of human BDCA2 and comprises a VH domain that is at least 70%, 75%, 80%, 85%, 90%, 91%, 92%, 93%, 94%, 95%, 96%, 97%, 98%, 99% or more identical to the amino acid sequence of the VH domain of BIIB059 (SEQ ID NO:7), or differs at least at 1 to 5 amino acid residues, but at fewer than 40, 30, 20, 15, or 10, residues, from SEQ ID NO:7. In certain instances, these antibodies (i) bind human or cynomolgus monkey BDCA2 but do not significantly bind BDCA2 from phylogenetic species below primates; and/or (ii) inhibit TLR7/TLR9-induced type I interferon and other cytokine or chemokine production by human pDCs; and/or (iii) mediate internalization of BDCA2 from the surface of pDCs; and/or (iv) downregulate CD32a and/or CD62L from the surface of pDCs; and/or (v) deplete pDCs in vitro by ADCC or CDC.

In certain embodiments, the anti-BDCA2 antibody or BDCA2-binding fragment thereof comprises a VL having the amino acid sequence set forth in SEQ ID NO:8. In some embodiments, the anti-BDCA2 antibody or antigen-binding fragment thereof selectively binds to the ectodomain of human BDCA2 and comprises a VL domain that is at least 70%, 75%, 80%, 85%, 90%, 91%, 92%, 93%, 94%, 95%, 96%, 97%, 98%, 99% or more identical to the amino acid sequence of the VL domain of BIIB059 (SEQ ID NO:8), or differs at least at 1 to 5 amino acid residues, but at fewer than 40, 30, 20, 15, or residues, from SEQ ID NO:8. In certain instances, these antibodies (i) bind human or cynomolgus monkey BDCA2 but do not significantly bind BDCA2 from phylogenetic species below primates; and/or (ii) inhibit TLR7/TLR9-induced type I interferon and other cytokine or chemokine production by human pDCs; and/or (iii) mediate internalization of BDCA2 from the surface of pDCs; and/or (iv) downregulate CD32a and/or CD62L from the surface of pDCs; and/or (v) deplete pDCs in vitro by ADCC or CDC.

In some embodiments, the anti-BDCA2 antibody or BDCA2-binding fragment thereof comprises a VH having the amino acid sequence set forth in SEQ ID NO:7 and a VL having the amino acid sequence set forth in SEQ ID NO:8. In some embodiments, the anti-BDCA2 antibody or antigen-binding fragment thereof selectively binds to the ectodomain of human BDCA2 and comprises (i) a VH domain that is at least 70%, 75%, 80%, 85%, 90%, 91%, 92%, 93%, 94%, 95%, 96%, 97%, 98%, 99% or more identical to the amino acid sequence of the VH domain of BIIB059 (SEQ ID NO:7), and (ii) a VL domain that is at least 70%, 75%, 80%, 85%, 90%, 91%, 92%, 93%, 94%, 95%, 96%, 97%, 98%, 99% or more identical to the amino acid sequence of the VL domain of BIIB059 (SEQ ID NO:8); or differs at least at 1 to 5 amino acid residues, but at fewer than 40, 30, 20, 15, or 10, residues, from SEQ ID NO:7 and/or SEQ ID NO:8. In certain instances, these antibodies (i) bind human or cynomolgus monkey BDCA2 but do not significantly bind BDCA2 from phylogenetic species below primates; and/or (ii) inhibit TLR7/TLR9-induced type I interferon and other cytokine or chemokine production by human pDCs; and/or (iii) mediate internalization of BDCA2 from the surface of pDCs; and/or (iv) downregulate CD32a and/or CD62L from the surface of pDCs; and/or (v) deplete pDCs in vitro by ADCC or CDC.

An antibody consisting of the mature heavy chain (SEQ ID NO:9) and the mature light chain (SEQ ID NO:10) listed below is termed “BIIB059” as used herein.

Mature BIIB059 heavy chain (HC)
(SEQ ID NO: 9)
DVQLVESGGG LVKPGGSLRL SCAAS [symbol] TYTMSWVRQA
PGKGLEWVAT ISPGDSFGYY YPDSVQGRFT ISRDNAKNSL
YLQMNSLRAE DTAVYYCTRD IYYNYGAWFA YWGQGTLVTV
SSASTKGPSV FPLAPSSKST SGGTAALGCL VKDYFPEPVT
VSWNSGALTS GVHTFPAVLQ SSGLYSLSSV VTVPSSSLGT
QTYICNVNHK PSNTKVDKKV EPKSCDKTHT CPPCPAPELL
GGPSVFLFPP KPKDTLMISR TPEVTCVVVD VSHEDPEVKF
NWYVDGVEVH NAKTKPREEQ YNSTYRVVSV LTVLHQDWLN
GKEYKCKVSN KALPAPIEKT ISKAKGQPRE PQVYTLPPSR
DELTKNQVSL TCLVKGFYPS DIAVEWESNG QPENNYKTTP
PVLDSDGSFF LYSKLTVDKS RWQQGNVFSC SVMHEALHNH
YTQKSLSLSP G
Mature BIIB059 light chain (LC)
(SEQ ID NO: 10)
DIQLTQSPSS LSASVGDRVT ITCKASQSVD YDGDSYMNWY
QQKPGKAPKL LIYAASTLES GVPSRFSGSG SGTDFTLTIS
SLQPEDFATY YCQQANEDPR TFGQGTKVEI KRTVAAPSVF
IFPPSDEQLK SGTASVVCLL NNFYPREAKV QWKVDNALQS
GNSQESVTEQ DSKDSTYSLS STLTLSKADY EKHKVYACEV
THQGLSSPVT KSFNRGEC

In the above VH, VL, HC, and LC sequences, CDRs 1, 2, and 3 based on the Kabat definition are both underlined and boldened. The italicized and boldened sequence in the VH and HC is the additional N-terminal sequence found in the CDR1 based on enhanced Chothia/AbM definition.

In certain embodiments, the anti-BDCA2 antibody or BDCA2-binding fragment thereof comprises a HC having the amino acid sequence set forth in SEQ ID NO:9. In some embodiments, the anti-BDCA2 antibody or antigen-binding fragment thereof selectively binds to the ectodomain of human BDCA2 and comprises a HC that is at least 70%, 75%, 80%, 85%, 90%, 91%, 92%, 93%, 94%, 95%, 96%, 97%, 98%, 99% or more identical to the amino acid sequence of SEQ ID NO:9, or differs at least at 1 to 5 amino acid residues, but at fewer than 40, 30, 20, 15, or 10, residues, from SEQ ID NO:9.

In certain embodiments, the anti-BDCA2 antibody or BDCA2-binding fragment thereof comprises a LC having the amino acid sequence set forth in SEQ ID NO:10. In some embodiments, the anti-BDCA2 antibody or antigen-binding fragment thereof selectively binds to the ectodomain of human BDCA2 and comprises a LC that is at least 70%, 75%, 80%, 85%, 90%, 91%, 92%, 93%, 94%, 95%, 96%, 97%, 98%, 99% or more identical to the amino acid sequence of SEQ ID NO:10, or differs at least at 1 to 5 amino acid residues, but at fewer than 40, 30, 20, 15, or 10, residues, from SEQ ID NO:10.

In some embodiments, the anti-BDCA2 antibody or BDCA2-binding fragment thereof comprises a HC having the amino acid sequence set forth in SEQ ID NO:9 and a LC having the amino acid sequence set forth in SEQ ID NO:10. In some embodiments, the anti-BDCA2 antibody or antigen-binding fragment thereof selectively binds to the ectodomain of human BDCA2 and comprises (i) a HC that is at least 70%, 75%, 80%, 85%, 90%, 91%, 92%, 93%, 94%, 95%, 96%, 97%, 98%, 99% or more identical to the amino acid sequence of SEQ ID NO:9, and (ii) a LC that is at least 70%, 75%, 80%, 85%, 90%, 91%, 92%, 93%, 94%, 95%, 96%, 97%, 98%, 99% or more identical to the amino acid sequence of SEQ ID NO:10; or differs at least at 1 to 5 amino acid residues, but at fewer than 40, 30, 20, 15, or 10, residues, from SEQ ID NO:9 and/or SEQ ID NO:10.

In certain embodiments, the anti-BDCA2 antibody is an IgG antibody. In specific embodiments, the anti-BDCA2 antibody has heavy chain constant region chosen from, e.g., IgG1, IgG2, IgG3, IgG4, IgM, IgA1, IgA2, IgD, and IgE. In one embodiment, the anti-BDCA2 antibody is of the IgG1 isotype. In another embodiment, the anti-BDCA2 antibody is of the IgG2 isotype. In yet another embodiment, the anti-BDCA2 antibody is of the IgG3 isotype. In further embodiments, the antibody has a light chain constant region chosen from, e.g., a human kappa or human lambda light chain. In a certain embodiment, the anti-BDCA2 antibody is an IgG1/kappa antibody. In certain embodiments, the anti-BDCA2 antibody includes a human Fc region that binds FcγRIIa (CD32a) with an EC₅₀ of 7 to 15 μg/mL. In certain embodiments, the antibody includes a human Fc region that binds FcγRIIa (CD32a) with an EC₅₀ of 10 μg/mL. In certain embodiments, the antibody includes a human Fc region that binds FcγRIIa (CD32a) with an EC₅₀ of 11 μg/mL. In certain embodiments, the antibody includes a human Fc region that binds FcγRIIa (CD32a) with an EC50 of 12 μg/mL. In some cases, the heavy chain constant region is human or a modified form of a human constant region. In certain instances the human constant region may include at least 1 and up to 2, 3, 4, 5, 6, 7, 8, 9, 11, 12, 13, 14, 15, 16, 17, 18, 19, or 20 substitutions. In a particular embodiment, the modified human Fc region is a modified human IgG1 Fc region. In some cases, the constant region of an anti-BDCA2 antibody may be modified by mutation of one or more amino acid residues to impart a desired functional property (e.g., altered effector function or half-life, reduced glycosylation). For example, the N-linked glycosylation site may be substituted to prevent or reduce N-linked glycosylation of Fc region (e.g., human IgG1 Fc region).

In some embodiments, the anti-BDCA2 antibody is a full-length (whole) antibody or substantially full-length. The protein can include at least one, and preferably two, complete heavy chains, and at least one, and preferably two, complete light chains. In some embodiments, the anti-BDCA2 antibody is a BDCA2-binding fragment. In some instances, the BDCA2-binding fragment is a Fab, a Fab′, an F(ab′)₂, a Facb, an Fv, a single chain Fv (scFv), a sc(Fv)2, or a diabody.

Antibodies, such as BIIB059, or BDCA2-binding fragments thereof can be made, for example, by preparing and expressing synthetic genes that encode the recited amino acid sequences or by mutating human germline genes to provide a gene that encodes the recited amino acid sequences. Moreover, this antibody and other anti-BDCA2 antibodies can be produced, e.g., using one or more of the following methods.

### Anti-BDCA2 Antibody Compositions

This disclosure also provides compositions (e.g., pharmaceutical compositions) comprising the anti-BDCA2 antibodies described herein. For example, the anti-BDCA2 antibody compositions comprises an anti-BDCA2 antibody comprising an immunoglobulin heavy chain variable domain (VH) and an immunoglobulin light chain variable domain (VL), wherein the VH comprises the VH-CDRs and the VL comprises the VL-CDRs of BIIB059. In certain instances, the VH-CDRs of comprise or consist of the amino acid sequences set forth in SEQ ID NO:1 or 17, SEQ ID NO:2, and SEQ ID NO:3; and the VL-CDRs comprise or consist of the amino acid sequences set forth in SEQ ID NO:4, SEQ ID NO:5, and SEQ ID NO:6. In some embodiments, the anti-BDCA2 antibody compositions comprises an anti-BDCA2 antibody comprising (i) a VH comprising or consisting of an amino acid sequence that is at least 85%, 90%, 91%, 92%, 93%, 94%, 95%, 96%, 97%, 98%, 99%, or 100% identical to the amino acid sequence set forth in SEQ ID NO:7; and (ii) a VL comprising or consisting of an amino acid sequence that is at least 85%, 90%, 91%, 92%, 93%, 94%, 95%, 96%, 97%, 98%, 99%, or 100% identical to the amino acid sequence set forth in SEQ ID NO:8. In certain embodiments, the anti-BDCA2 antibody compositions comprises an anti-BDCA2 antibody comprising (i) a heavy chain comprising or consisting of an amino acid sequence that is at least 85%, 90%, 91%, 92%, 93%, 94%, 95%, 96%, 97%, 98%, 99%, or 100% identical to the amino acid sequence set forth in SEQ ID NO:9; and (ii) a light chain comprising or consisting of an amino acid sequence that is at least 85%, 90%, 91%, 92%, 93%, 94%, 95%, 96%, 97%, 98%, 99%, or 100% identical to the amino acid sequence set forth in SEQ ID NO:10. The pharmaceutical composition can further include any pharmaceutically acceptable carrier and/or excipient.

In some cases, the pharmaceutical composition comprises the anti-BDCA2 antibody at a concentration of 150 mg/ml; sucrose at a concentration of 3%; L-histidine at a concentration of 20 mM; L-Arginine HCl at a concentration of 100 mM; glutathione (GSH or a combination of GSH and GSSG) at a concentration of 0.4 mM; and polysorbate 80 (PS80) at a concentration of 0.05%. In some cases, the pharmaceutical composition has a pH of 5.5 to 6.0. In one instance, the pharmaceutical composition has a pH of 5.7.

In some cases, the pharmaceutical composition comprises the anti-BDCA2 antibody at a concentration of 100-200 mg/ml; sucrose at a concentration of 1-4%; L-histidine at a concentration of 10-30 mM; L-Arginine HCl at a concentration of 75-150 mM; glutathione (GSH or a combination of GSH and GSSG) at a concentration of 0.2 to 0.6 mM; and polysorbate 80 (PS80) at a concentration of 0.02% to 0.08%. In some cases, the pharmaceutical composition has a pH of 5.5 to 6.0. In one instance, the pharmaceutical composition has a pH of 5.7.

In some cases, the pharmaceutical composition is a pharmaceutical composition described in US20190284281, which is incorporated herein by reference.

In some instances, the compositions described herein may be formulated with, or be administered along with, at least one of an antimalarial, a corticosteroid, an immunosuppressive drug, or an anti-B-lymphocyte stimulator (BLyS) monoclonal antibody. In certain instances, compositions of this disclosure may be formulated with, or be administered along with, at least one of a mycophenolate, an azathioprine, methotrexate, a calcineurin inhibitor, or cyclophosphamide. In one instance, the compositions described herein are administered along with an antimalarial. In another instance, the compositions described herein are administered along with a corticosteroid.

The antibody or the pharmaceutical composition can be provided in a pre-filled syringe or pump. The pharmaceutical composition comprises a sterile preparation of an anti-BDCA2 antibody described herein. The pre-filled syringe or pump can be adapted for subcutaneous administration of the anti-BDCA2 antibody at a fixed dose of 225 mg.

The compositions of this disclosure may be packaged as a kit with information regarding how the antibody is to be used for the treatment of CLE or SLE. The kit may include at least one of an antimalarial, a corticosteroid, an immunosuppressive drug, or an anti-B-lymphocyte stimulator (BLyS) monoclonal antibody. In certain instances, the kit can include at least one of a mycophenolate, an azathioprine, methotrexate, a calcineurin inhibitor, or cyclophosphamide. In one instance, the kit includes an antimalarial. In another instance, the kit includes a corticosteroid.

### Methods of Treatment

An anti-BDCA2 antibody described herein, or a pharmaceutical composition comprising an anti-BDCA2 antibody described herein, can be used to treat or prevent a variety of immunological disorders, such as inflammatory and autoimmune disorders (e.g., CLE, SLE, lupus nephritis, neuropsychiatric lupus (NPSLE), Sjogren syndrome, systemic sclerosis (scleroderma), morphea, psoriasis, rheumatoid arthritis, inflammatory bowel disease (IBD), dermatomyositis, polymyositis, type I diabetes, or cytokine release syndrome). Based on in vitro data, anti-BDCA2 antibodies can disable and/or inhibit inflammatory cytokines and chemokines produced by pDCs, downregulate CD32a, inhibit immune complex stimulation of pDCs, and/or downregulate or cause shedding of CD62L. The anti-BDCA2 antibodies or BDCA2-binding fragment thereof of this disclosure can be combined with an antimalarial agent (e.g., HCQ) for potential improved therapeutic effects in the treatment of inflammatory and autoimmune disorders. Anti-BDCA2 antibodies can be used to reduce levels of cytokines and chemokines such as: type I interferons, type III interferons, IL-6, TNF-α, MIP1-α and MIP1-β, CCLS, and IP-10. Type I IFNs constitute a multiple-member family of cytokines, including 13 IFN-α subtypes, IFN-β, -ε, -κ, -ω, -δ and -τ. (Theofilopoulos, Annu. Rev. Immunol., 23:307-36 (2005)). Type III interferons consist of three IFN-λ molecules called IFN-λ1, IFN-λ2 and IFN-λ3 (also referred to as IL29, IL28A and IL28B, respectively). By depleting and/or dampening pDC function, the anti-BDCA2 antibodies described herein provide a more robust treatment approach than treatments attempting to reduce specific IFN subtypes with neutralizing antibodies. In addition, the pDC-specific treatment approach of the anti-BDCA2 antibodies is more selective and potentially safer than global blockade of the IFN response. For example, anti-BDCA2 antibodies described herein effectively eliminate pDC-derived type I IFNs while maintaining other sources of IFN that could be necessary in the event of viral infections.

In one instance, an anti-BDCA2 antibody described herein, or a pharmaceutical composition comprising an anti-BDCA2 antibody described herein, is used to treat CLE in a human subject in need thereof. In some cases, the CLE disease is mild CLE activity. In some instances, the CLE disease is moderate CLE activity. In other instances, the CLE disease is severe CLE activity. In some cases, the CLE disease is acute CLE (ACLE). In some cases, the CLE disease is subacute CLE (SCLE). In some cases, the CLE disease is chronic CLE (CCLE). In certain cases, the CLE disease is discoid lupus erythematosus (DLE). In some cases, the CLE disease is active CLE. In some cases, the CLE disease is active CLE and the human subject is intolerant and/or refractory to antimalarial and topical steroid therapy. In certain cases, the active CLE disease is CLE with systemic manifestations of lupus and the human subject is intolerant or refractory to antimalarial and/or topical steroid therapy. In some cases, the active CLE disease is CLE without systemic manifestations of lupus and the human subject is intolerant or refractory to antimalarial and/or topical steroid therapy. In certain instances, the human subject achieves clinically meaningful reduction, e.g. a 4-point decrease, in Cutaneous Lupus Erythematosus Disease Area and Severity Index-A (CLASI-A) score from baseline about sixteen weeks to about 24 weeks after the first administration of the anti-BDCA2 antibody. See Albrecht et al., “The CLASI (Cutaneous Lupus Erythematosus Disease Area and Severity Index): an outcome instrument for cutaneous lupus erythematosus” J Invest Dermatol. 2005; 125(5):889-94; Bonilla-Martinez et al., “The cutaneous lupus erythematosus disease area and severity index: a responsive instrument to measure activity and damage in patients with cutaneous lupus erythematosus” Arch Dermatol. 2008; 144(2):173-80; Klein et al., “Using the CLASI to assess disease severity and responsiveness to therapy in cutaneous lupus erythematosus” Arthritis Rheumatism. 2009; 60:903; Klein et al., “Development of the CLASI as a tool to measure disease severity and responsiveness to therapy in cutaneous lupus erythematosus” Arch Dermatol. 2011; 147(2):203-8; and Chakka et al., “Evaluating change in disease activity needed to reflect meaningful improvement in quality of life for clinical trials in cutaneous lupus erythematosus” J Am Acad Dermatol. 2021 June; 84(6):1562-1567. In some cases, the human subject achieves a clinically meaningful reduction from baseline in disease activity on an IGA specific for CLE, e.g., a score of 0, 1, 2, or 3, about sixteen weeks to about 24 weeks after the first administration of the anti-BDCA2 antibody.

In another instance, an anti-BDCA2 antibody described herein, or a pharmaceutical composition comprising an anti-BDCA2 antibody described herein, is used to treat SLE in a human subject in need thereof. In certain instances, the SLE is active SLE. In some cases, the human subject has active, autoantibody-positive SLE. In certain cases, wherein the human subject has active, autoantibody-positive SLE and the human subject is receiving the standard of care therapy for SLE. In some cases, the SLE is moderate SLE. In certain cases, the SLE is severe SLE. In some cases, the SLE is active SLE with active joint and/or skin manifestations. In some cases, the human subject has a SLEDAI-2K≥6 excluding alopecia, lupus-related headache and organic brain disease at initiation of treatment. In certain cases, the human subject has a clinical SLEDAI-2K≥4 excluding alopecia, lupus-related headache and organic brain disease, anti-ds DNA, low complement C3 and/or C4, or fever, at initiation of treatment. In certain cases, the human subject has BILAG-2004 grade A in ≥1 organ system or BILAG-2004 grade B in ≥2 organ systems at initiation of treatment. In certain instances, the human subject achieves an SRI-4 response about 3, 4, 5, 6, 7, 8, 9, 10, 12, 16, 24, or 52 weeks after initiation of treatment with the anti-BDCA2 antibody. In certain instances, the human subject achieves a Joint-50 response rate about 3, 4, 5, 6, 7, 8, 9, 12, 14, 16, 24, or 52 weeks after initiation of treatment with the anti-BDCA2 antibody in a human subject who had at least 4 joints that are swollen and tender at initiation of treatment. In certain instances, the human subject has a CLASI-A score ≥10 at baseline and achieves a CLASI-50 response about 3, 4, 5, 6, 7, 8, 9, 10, 12, 16, 24, or 52 weeks after initiation of treatment with the anti-BDCA2 antibody.

In some instances, the anti-BDCA2 antibody is administered to the human subject at a dose of 225 mg every four weeks via a subcutaneous injection. In some instances, the subject is administered at least 2, at least 3, at least 4, at least 5, at least 6, at least 7 at least 8, at least 9, at least 10 doses, at least 11 doses, at least 12 doses, or 2, 3, 4, 5, 6, 7, 8, 9, 10, 11, 12, 13, 14, 15, 16, 17, 18, 19, 20, 21, 22, 23, 24, 25, 26, 27, 28, 29, or 30 doses. In some instances, the subject is administered doses until the health care practitioner deems it unnecessary. In some instances, the subject is administered for the lifetime of the subject. In some cases, the subject is administered a loading dose of the anti-BDCA2 antibody about two weeks after the first administration of the anti-BDCA2 antibody. In some cases, the subject is administered a loading dose of the anti-BDCA2 antibody about two weeks, three weeks, four weeks, or five weeks after the first administration of the anti-BDCA2 antibody. In some cases, the loading dose is 225 mg. In some cases, the subject is administered a second loading dose of the anti-BDCA2 antibody 12, 13, 14, 15, 16, 17, 18, 19, 20, 21, 22, 23, or 24, 25, or 26 weeks (e.g., eighteen weeks) after the first administration of the anti-BDCA2 antibody. In some cases, the second loading dose is 225 mg.

In some cases, the method of treating the human subject is by way of administering an anti-BDCA2 antibody pharmaceutical composition. The pharmaceutical composition comprises the anti-BDCA2 antibody at a concentration of 150 mg/ml; sucrose at an a concentration of 3%; L-histidine at a concentration of 20 mM; L-Arginine HCl at a concentration of 100 mM; glutathione (GSH or a combination of GSH and GSSG) at a concentration of 0.4 mM; and polysorbate 80 (PS80) at a concentration of 0.05%. In some cases, the pharmaceutical composition has a pH of 5.5 to 6.0. In one instance, the pharmaceutical composition has a pH of 5.7. The pharmaceutical composition is administered subcutaneously every four weeks to provide a dose of 225 mg of the anti-BDCA2 antibody to the human subject.

In some embodiments in all of the above-described methods of treatment, the anti-BDCA2 antibody selectively binds to the ectodomain of human BDCA2 and comprises (i) a VH domain that is at least 70%, 75%, 80%, 85%, 90%, 91%, 92%, 93%, 94%, 95%, 96%, 97%, 98%, 99% or more identical to the amino acid sequence of the VH domain of BIIB059 (SEQ ID NO:7), and/or (ii) a VL domain that is at least 70%, 75%, 80%, 85%, 90%, 91%, 92%, 93%, 94%, 95%, 96%, 97%, 98%, 99% or more identical to the amino acid sequence of the VL domain of BIIB059 (SEQ ID NO:8); or differs at least at 1 to 5 amino acid residues, but at fewer than 40, 30, 20, 15, or 10, residues, from SEQ ID NO:7 and/or SEQ ID NO:8. In certain instances, these anti-BDCA2 antibodies (i) bind human or cynomolgus monkey BDCA2 but do not significantly bind BDCA2 from phylogenetic species below primates; and/or (ii) inhibit TLR7/TLR9-induced type I interferon and other cytokine or chemokine production by human pDCs; and/or (iii) mediate internalization of BDCA2 from the surface of pDCs; and/or (iv) downregulate CD32a and/or CD62L from the surface of pDCs; and/or (v) deplete pDCs in vitro by ADCC or CDC.

In certain embodiments in all of the above-described methods of treatment, the anti-BDCA2 antibody selectively binds to the ectodomain of human BDCA2 and comprises (i) a HC that is at least 70%, 75%, 80%, 85%, 90%, 91%, 92%, 93%, 94%, 95%, 96%, 97%, 98%, 99% or more identical to the amino acid sequence of SEQ ID NO:9, and/or (ii) a LC that is at least 70%, 75%, 80%, 85%, 90%, 91%, 92%, 93%, 94%, 95%, 96%, 97%, 98%, 99% or more identical to the amino acid sequence of SEQ ID NO:10; or differs at least at 1 to 5 amino acid residues, but at fewer than 40, 30, 20, 15, or 10, residues, from SEQ ID NO:9 and/or SEQ ID NO:10.

The following are examples of the practice of the invention. They are not to be construed as limiting the scope of the invention in any way.

### EXAMPLES

### Example 1: Pharmacokinetic Time-Course Study with BIIB059

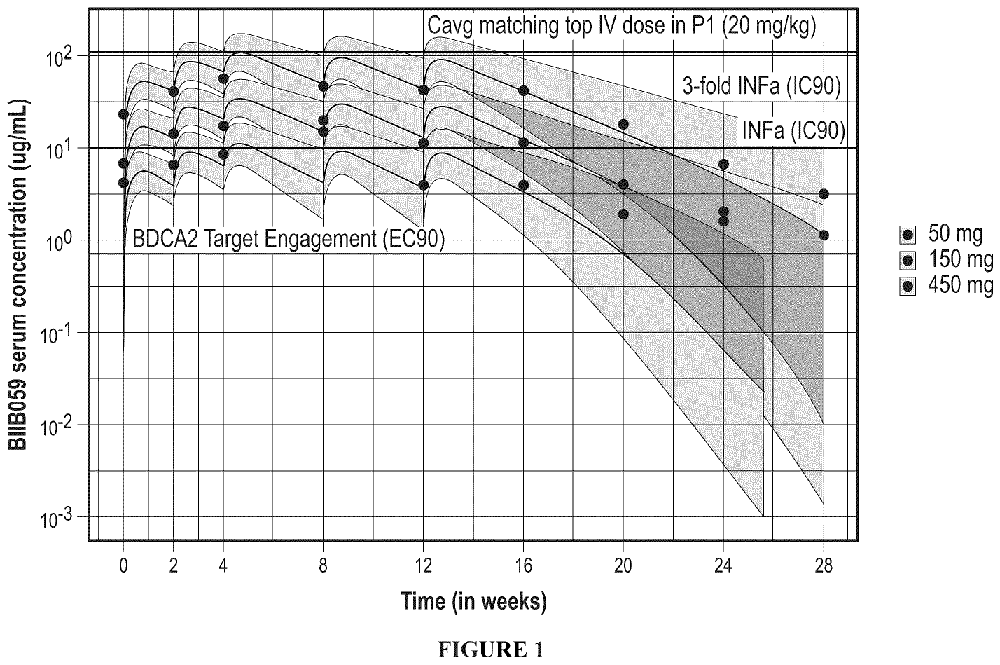

In a Phase 2 study of CLE, BIIB059 fixed doses of 50, 150 and 450 mg administered subcutaneously (SC) every 4 weeks (Q4W) were studied. A loading dose was administered at week 2 (for patients receiving 50 mg SC Q4W, the loading dose was also 50 mg; for patients receiving 150 mg SC Q4W, the loading dose was also 150 mg; and for patients receiving 450 mg SC Q4W, the loading dose was also 450 mg). As shown in FIG. 1, these three BIIB059 doses demonstrated distinct exposure with minimum overlap. The observed PK was consistent with the PK/PD model projections, indicating that the doses tested in this study were within the expected efficacious range of BIIB059. All doses were considered safe and well tolerated.

The primary endpoint at week 16 defined as CLASI-A score percent change from baseline was met (Table 1) and a dose-response was demonstrated.

TABLE 1
CLASI-A Score (Part B): Percent change from Baseline at Week
16, Mixed Model for Repeated Measures - MITT Population

| Week 16 | | | | | | | | |
|---|---|---|---|---|---|---|---|---|
| percent | | | | | | | | |
| change from | Placebo | | BIIB059 50 mg | | BIIB059 150 mg | | BIIB059 450 mg | |
| baseline | (N = 33) | | (N = 26) | | (N = 25) | | (N = 48) | |
| n | 31 | | 23 | | 24 | | 42 | |
| Mean (SD) | −15.03 | (37.23) | −35.52 | (33.35) | −47.11 | (34.10) | −41.66 | (37.33) |
| LS Mean (SE) | −14.49 | (6.43) | −38.78 | (7.51) | −47.91 | (7.47) | −42.48 | (5.50) |
| LS Mean | | | −24.29 | (−43.70, −4.88) | −33.42 | (−52.71, −14.12) | −27.99 | (−44.55, −11.42) |
| Difference | | | | | | | | |
| (95% CI) | | | | | | | | |
| p-value | | | 0.015 | | <0.001 | | 0.001 | |
| (BIIB059 vs | | | | | | | | |
| placebo) | | | | | | | | |

Note 1:
A Mixed Effect Model Repeat Measurement (MMRM) model is performed, using treatment group, study visit, study visit by treatment interaction, DLE (Yes/No), CLASIA score (<=10 vs. >10) as fixed effect covariates. The MMRM model used an unstructured covariance structure.
Note 2:
For subjects who are considered as treatment failures, worse of baseline or last visit before treatment failure carried forward is used to impute values for all the visits post treatment failure visit. Data after subjects' treatment discontinuation are censored.
Note 3:
For subjects from PV1 part B who have completed treatment up to week 12 but could not re-consent to PV2, Week 16 data of these patients was imputed using predicted value from the MMRM model for absolute value.
Abbreviations: Abbreviations: SD = standard deviation, SE = standard error, CI = confidence interval, MMRM = Mixed models repeated measures, LS = Square, MITT = Modified intent to treat.

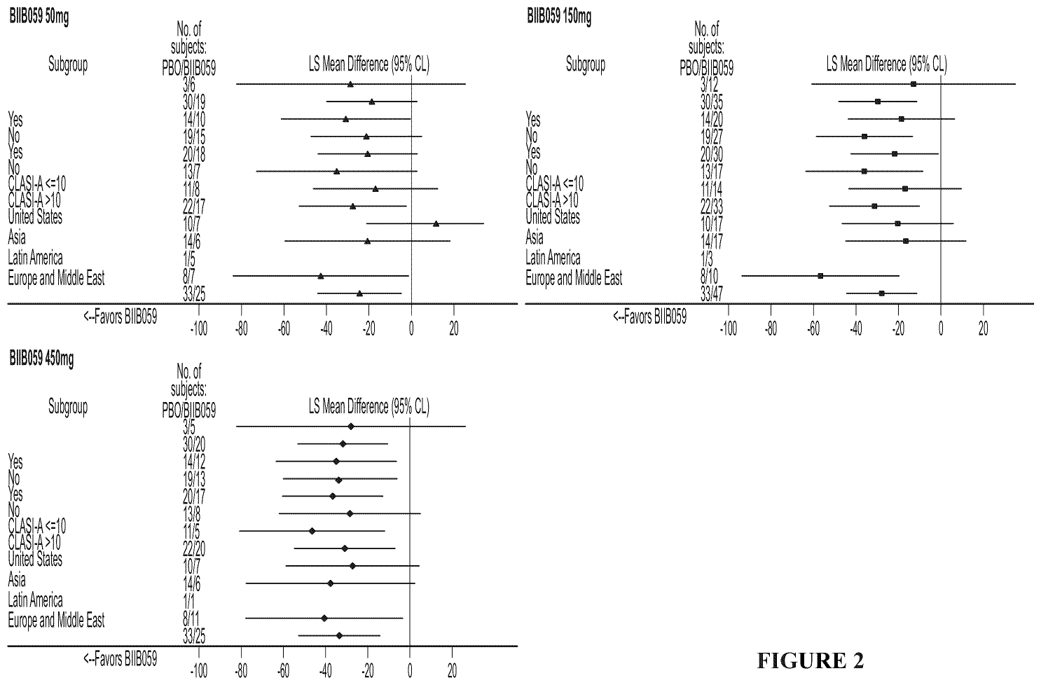

Statistical significance was achieved for the 150 mg and 450 mg doses. Efficacy of BIIB059 was also shown across subgroups, specifically with 150 mg and 450 mg doses, while the 50 mg dose showed more inconsistent clinical results (FIG. 2). Altogether, the Phase 2 data indicate that a dose ≥150 mg of BIIB059 is needed to achieve clinically meaningful improvements in the CLE skin disease activity across multiple thresholds and subgroups.

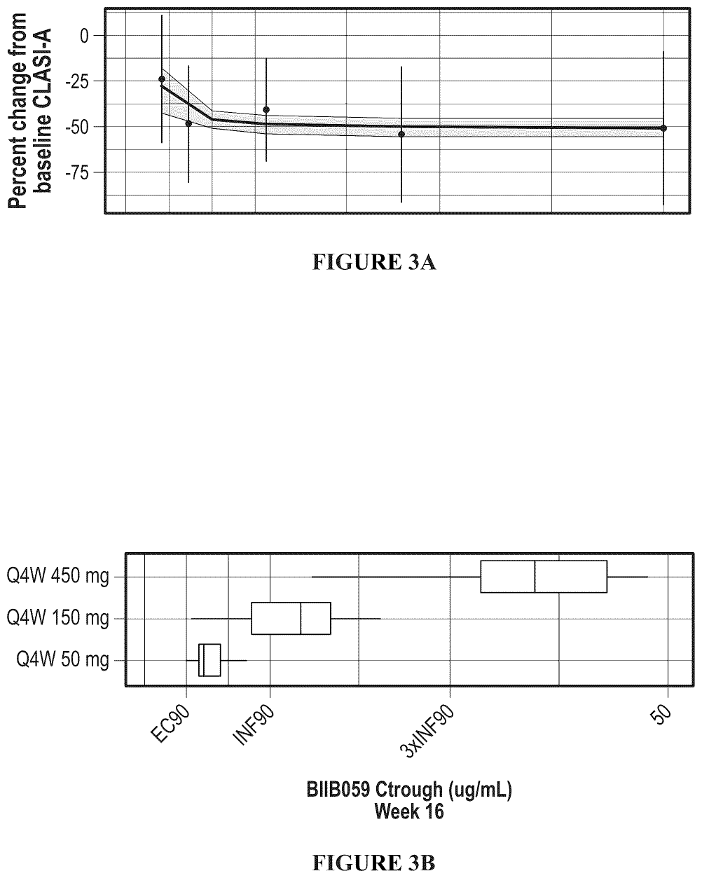

The relationship between BIIB059 serum exposure and its efficacy (assessed by the CLASI-A) in participants with CLE was characterized using data from the Phase 2 study. BIIB059's effect on the percent change from baseline of CLASI-A at week 16 was described using Emax function. Model predicted trough concentrations were used as an input for the exposure response analysis. The point estimate for BIIB059 concentration associated with 90% of the maximal effect (EC90) on the CLASI-A (10.1 ug/mL) was comparable to the IC90 for IFN-α (9.7 ug/mL). FIG. 3 shows the observed (±SD) and predicted (90% CI) change from baseline in CLASI-A at week 16 (primary endpoint) as a function of the BIIB059 trough concentrations in participants with CLE. The fraction of participants achieving CLASI-50 response increased with increasing BIIB059 exposures, with maximal efficacy estimated to be achieved at exposures corresponding to the 150 mg SC Q4W dose (approximately IFN-α IC90).

Of note, the 50 mg dose resulted in reduced efficacy, suggesting that beyond BDAC2 internalization, downstream regulation of the IFN-α level is needed in order to maximize the therapeutic benefit of BIIB059 in participants with CLE.

### Example 2: Dose Selection for Phase 3 Studies for CLE

Exposure-response models were used to simulate Q4W SC regimens of BIIB059. A dose of 225 mg Q4W SC is selected for Phase 3 studies based upon participant exposure levels achieving CLASI-A EC90 or higher.

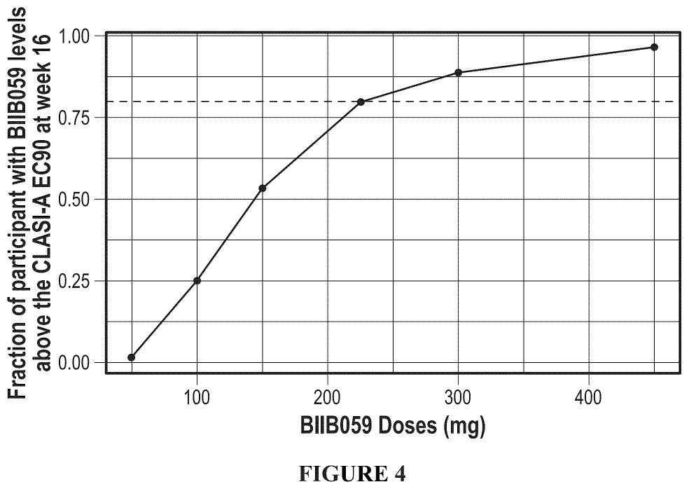

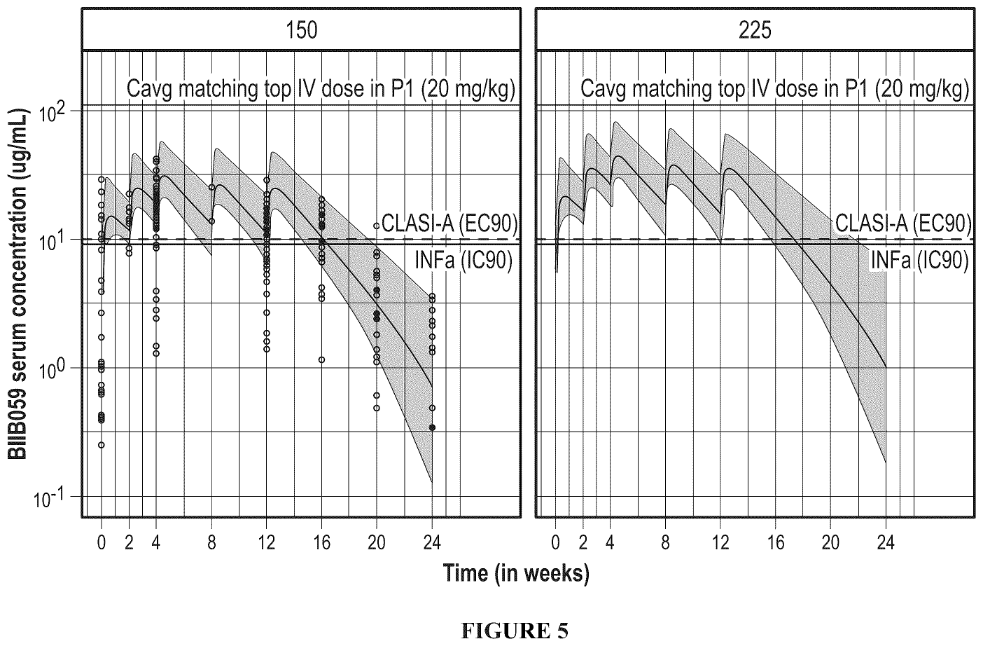

FIG. 4 is a model simulation showing the percent of participants with BIIB059 levels above 10.1 ug/mL (or CLASI-A EC90) and FIG. 5 is a model simulation showing the PK time-courses after 150 mg and 225 mg BIIB059 doses. The model simulations suggest that a higher proportion of the participants at the 225 mg dose would be above the EC90 relative to the 150 mg at week 16. At the 50 mg dose, less than 5% of the subjects had BIIB059 trough levels above the EC90, which resulted in lower efficacy at week 16 (FIG. 3). Based on exposure-response analyses of the Phase 2 study, a dose of 225 mg Q4W SC is expected to maximize the potential for efficacy of BIIB059 in participants with CLE. The exposure levels at trough concentration of the proposed Phase 3 dosage are expected to remain above the CLASI-A EC90 for at least 80% of the participants.

The impact of immunogenicity was also assessed on the BIIB059 exposure as related to the Phase 3 dose selection. In participants with CLE from the Phase 2 study, 5 participants (19.2%), 4 participants (16.0%), and 5 participants (10.4%) in the BIIB059 50, 150, and 450 mg treatment groups, respectively, were evaluated as positive for anti-BIIB059 antibodies. All participants in the placebo group were negative for anti-BIIB059 antibodies. As shown in FIG. 5, participants who were ADA-positive at the 150 mg dose in Phase 2 (filled dots in FIG. 5) had exposure levels consistently above the EC90 during the treatment phase. In Phase 3, assuming an ADA incidence rate of 19%, BIIB059 exposure is expected to remain above the efficacious levels for at least 80% of the participants dosed with 225 mg (shaded area in right hand panel of FIG. 5). This indicates that the impact of immunogenicity on exposure is not expected to translate into clinically meaningful differences on efficacy or safety.

In sum, BIIB059 225 mg Q4W SC is an appropriate dose for CLE, based on the safety, PK, PD (BDCA2 internalization), efficacy and extrapolated inhibitory potency (concentration resulting in 90% inhibition of response [IC90]) of pDC IFN-α production.

### Example 3: Phase 3 CLE Study Design

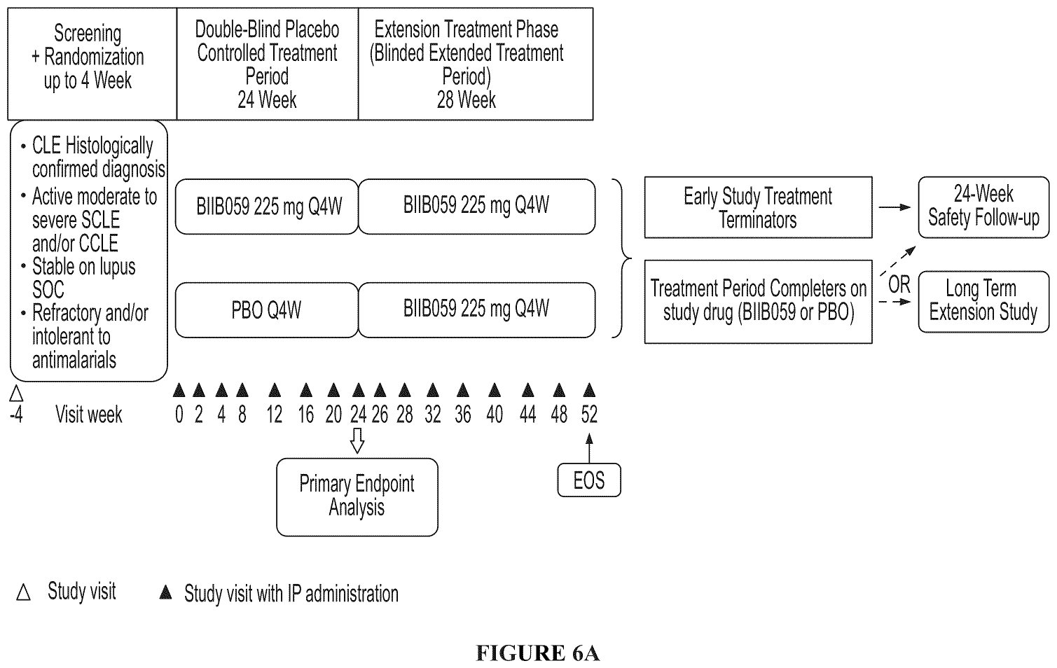

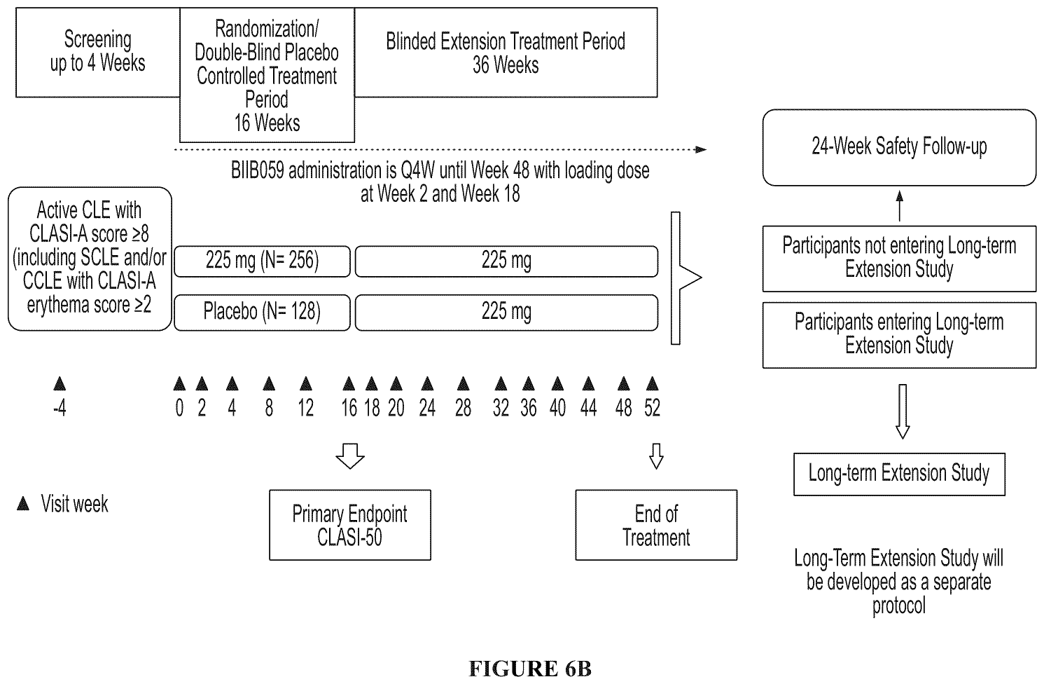

The study is a randomized, double-blind, placebo-controlled, multi-center, Phase 3 trial (FIG. 6A). Approximately 384 participants from 160 sites globally are enrolled and randomly assigned in a 2:1 ratio in each study to receive either BIIB059 225 mg Q4W SC or matching placebo, respectively, for a 24-week double-blind, placebo controlled (DBPC) treatment period. Subsequently, participants who complete the DBPC treatment period continue in a 28-week blinded extended (BE) treatment period, during which all participants receive BBB059 225 mg SC Q4W and the blinding to the initial treatment assignment is maintained.

Study participants have active CLE with or without systemic manifestations and are refractory and/or intolerant to antimalarial. The diagnosis of CLE is histologically confirmed in the past or at screening. The disease activity is defined by CLASI-A. All participants must have active cutaneous manifestations defined as: an overall CLASI-A score ≥10 and a CLA-IGA-R score ≥3, adjudicated at screening and confirmed at randomization, and at least 1 SCLE lesion with a minimum CLASI-A erythema score of ≥2 and CLASI-A scaling score ≥1, and/or at least one active COLE lesion with a minimum CLASI-A erythema score of ≥2 and CLASI-D scarring score ≥1, a CLA-IGA-R erythema score ≥3 and a score ≥1 in the 4 morphological characteristics jointly of the CLA-IGA-R (scale, edema, follicular involvement, or secondary change). Participants with concurrent active ACLE in addition to active SCLE and/or COLE lesions, with or without SLE, are allowed in the studies. In addition, participants at screening must have documentation of current failure to respond to antimalarial treatment used for ≥12 weeks or previously documented discontinuation of antimalarial due to poor tolerability and/or side effect and/or lack of therapeutic effect after 12 weeks of use. Other standard of care lupus treatments, such as but not restricted to oral corticosteroids, mycophenolate or azathioprine are allowed within the specifications described in the protocol and should have been initiated at least 12 weeks prior to randomization. Any treatments received at randomization must remain stable for the duration of the study.

The objective of this Phase 3 study is to confirm the efficacy and safety of BIIB059 in participants with active CLE with or without systemic manifestations, who are intolerant or refractory to antimalarials treatment.

This study will implement regionally-defined primary and key secondary endpoints. One set of endpoints are defined for the US and another set of endpoints for the rest of world (ROW).

The primary objective will be to demonstrate the efficacy of BBB059 in reducing CLE disease activity evaluated by CLA-IGA-R score of 0 or 1 response rate, defined as clear or almost clear skin disease activity, in BIIB059 treated participants versus placebo treated participants at Week 16 in the US, and by CLASI-70 responder rate in BIIB059 treated participants versus placebo treated participants at Week 24 in ROW. CLASI-70 is defined as at least a 70% reduction in CLASI-A score from baseline (Table 2).

TABLE 2
CLE Phase 3 Study: Primary and Key Secondary Endpoints
Study Objectives and Endpoints for Part B (Phase 3) -- US

| Primary Objective | Primary Endpoint |
|---|---|
| To demonstrate the efficacy of BIIB059 | Proportion of participants who achieve a |
| compared with placebo in reducing skin | CLA-IGA-R score of 0 or 1, defined as |
| disease activity measured by the | clear or almost clear skin disease activity |
| CLA-IGA-R in participants with active | at Week 16. |
| SCLE and/or CCLE with or without | |
| systemic manifestations and refractory | |
| and/or intolerant to antimalarials. | |
| | Key Secondary Endpoints (Multiplicity |
| Key Secondary Objectives | Adjusted) |
| To demonstrate the efficacy of BIIB059 | Proportion of participants who achieve a |
| compared with placebo in reducing skin | score of 0 or 1 in the CLA-IGA-R |
| disease activity of erythema or | erythema characteristic at Week 16. |
| 4 morphological characteristics jointly | Proportion of participants who achieve at |
| measured by CLA-IGA-R in participants | least 1 level improvement in the |
| with active SCLE and/or CCLE with or | CLA-IGA-R 4 morphological |
| without systemic manifestations and | characteristic jointly at Week 16. |
| refractory and/or intolerant to antimalarials. | |
| Hypothesis: BIIB059 improves skin | |
| disease activity of CLE in achieving | |
| clear or almost clear skin condition in | |
| erythema as measured by CLA-IGA-R | |
| in all participants, compared with | |
| placebo and standard of care (SoC). | |
| To demonstrate the efficacy of BIIB059 | Proportion of participants who achieve a |
| compared with placebo in reducing skin | CLASI-70 response, defined as ≥70% |
| disease activity measured by CLASI-A | decrease in baseline CLASI-A score at |
| score in participants with active SCLE | Week 16. |
| and/or CCLE with or without systemic | |
| manifestations and refractory and/or | |
| intolerant to antimalarials. | |

Study Objectives and Endpoints for Part B (Phase 3) - ROW

| Primary Objective | Primary Endpoint |
|---|---|
| To demonstrate the efficacy of BIIB059 | Proportion of participants who achieve |
| compared with placebo in reducing skin | CLASI-70 response, defined as ≥70% |
| disease activity measured by CLASI-A | decrease in CLASI-A score from |
| score in participant. with active SCLE | baseline to Week 24. |
| and/or CCLE with or without systemic | |
| manifestations and refractory and/or | |
| intolerant to antimalarials. | |
| Key Secondary Objectives | Key Secondary Endpoints |
| To evaluate the efficacy of BIIB059 | The proportion of participants who |
| compared with placebo to achieve | achieve a CLA-IGA-R score of 0 or 1, |
| clinically relevant improvement in skin | defined as clear or almost clear skin |
| disease activity measured by the | disease activity at Week 24. |
| CLA-IGA-R. | |
| To demonstrate the efficacy of BIIB059 | Proportion of participants who achieve a |
| compared to placebo in reducing skin | score of 0 or 1 in the CLA-IGA-R |
| disease activity measured by CLA-IGA-R | erythema characteristic at Week 24. |
| erythema characteristic or 4 morphological | Proportion of participants who achieve at |
| characteristics jointly in participants. with | least 1 level of improvement in the |
| active SCLE and/or CCLE with or without | 4 morphological characteristics jointly at |
| systemic manifestations and refractory | Week 24. |
| and/or intolerant to antimalarials | |

Key secondary objectives (Table 2) will evaluate the efficacy of BIIB059 225 mg Q4W SC compared with placebo in reducing skin disease activity measured by the CLA-IGA-R score of 0 to 1, CLA-IGA-R erythema characteristic or 4 morphological characteristics jointly in participants at week 16 in the US and at week 24 in the ROW, and by CLASI-70 response rate at week 16 in the US.

Key secondary endpoints supporting secondary objectives are shown in Table 2.

Eligible participants who complete the treatment period through Week 52 on study drug, will be offered to enter a long term extension study (LTE). Participants who prematurely discontinue study participation or study medication, and participants who complete the blinded extended treatment period but choose to not participate in the LTE will complete a 24-week safety follow up.

The LTE study will be a multi-center, open label, interventional study that will enroll about 400-500 participants from the parental Phase 3 studies. The LTE study is planned to further evaluate the safety and efficacy profile of BIIB059 for at least an additional 2 years of follow-up. The eligible population would include all participants completing the two 52-week phase 3 studies who consent to continue. The primary objective of the LTE study will be to evaluate the long-term safety (overall exposure and frequencies of adverse events [AE]) of BIIB059. The proposed dose of BIIB059 225 mg Q4W SC in the LTE study is identical to the one planned for use in the BIIB059 phase 3 study.

An alternate schematic diagram of a CLE study design is depicted in FIG. 6B.

### Example 4: Dose Selection for Phase 3 Studies for SLE

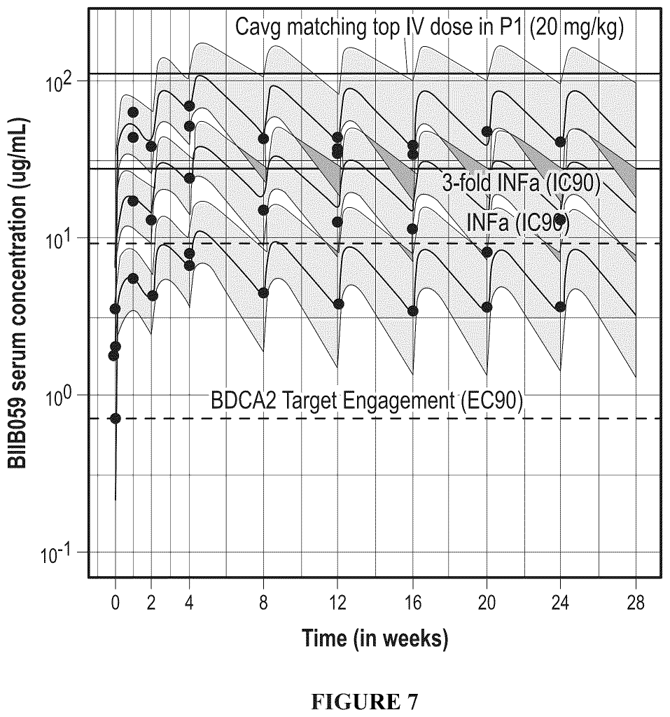

In a Phase 2 study of SLE, BIIB059 fixed doses of 50, 150 and 450 mg administered subcutaneously (SC) every 4 weeks (Q4W) were studied. As shown in FIG. 7, the 50, 150, and 450 mg doses demonstrated distinct exposure with minimum overlap in SLE. The observed PK was consistent with the PK/PD model projections, indicating that the doses tested in SLE in Phase 2 were within the therapeutic range of BIIB059. All doses were considered safe and well tolerated.

Exposure response (E-R) analyses were performed to characterize the relationships between BIIB059 exposure and efficacy measured through the SLE responder Index-4 (SRI-4). The E-R models were subsequently used to perform clinical trial simulations to support dosage regimen selections for Phase 3 in participants with active SLE.

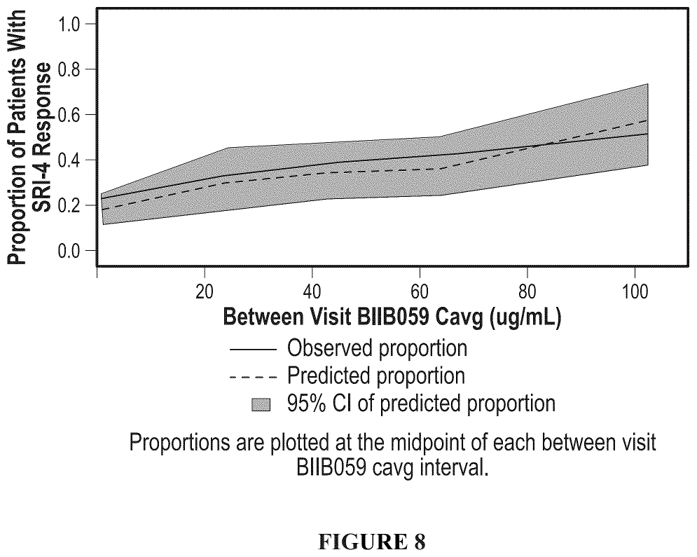

The SRI-4 model was developed using data from the study, which included placebo (43%) and mostly active treatment patients administered BIIB059 450 mg (48%). The remaining 9% of the data was equally distributed among the lower doses of 50 and 150 mg. The E-R model for the probability of SRI 4 was a logistic regression model including a power time course plus a piece wise linear function of BIIB059 Cavg (fixed to 55 μg/mL based on sensitivity analysis). FIG. 8 provides the predicted proportion (95% CI) of patients with SRI 4 from simulated datasets (dotted line) overlaid on the observed data (solid line) versus BIIB059 Cavg.

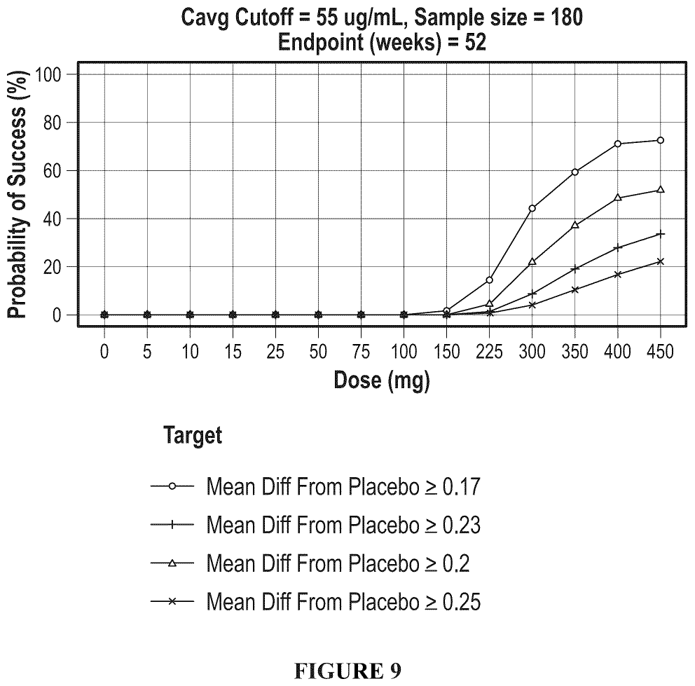

Clinical trial simulations were performed using the final E-R models for SRI 4 responses to inform the dose selection in Phase 3 for participants SLE. Higher BIIB059 doses resulted in a higher probability of success to achieve a significant treatment difference in SRI 4 response relative to placebo (FIG. 9).

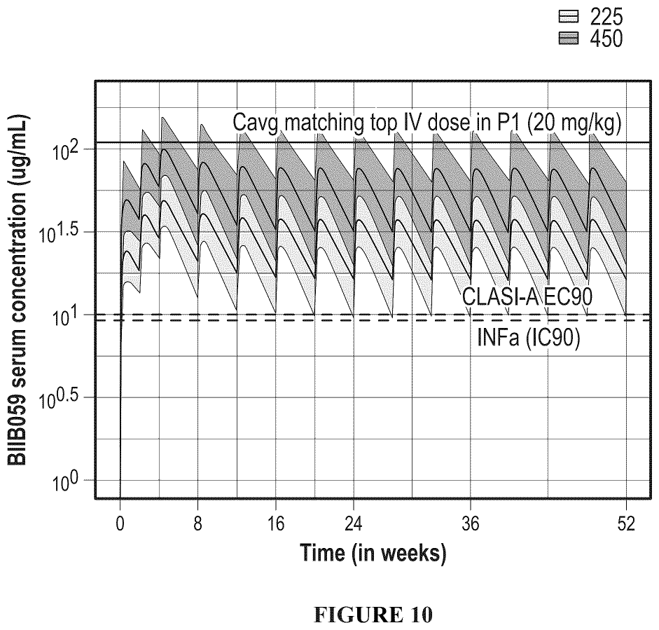

BIIB059 doses of 225 mg (low dose) and 450 mg (high dose) SC Q4W, with an additional dose at Week 2, are proposed for this Phase 3 study. The exposure associated with doses ranging from 50 mg to 450 mg Q4W (with an additional dose at Week 2) was deemed to be well tolerated among SLE and/or CLE participants in previous clinical studies. The exposure levels at trough concentration of the 225 mg dose are expected to remain consistently above the IC90 for IFN-α associated with the efficacy of BIIB059 in Phase 2 (FIG. 10) and account for an immunogenicity rate of up to 19% observed in Phase 2, which is not expected to impact the efficacy.

### Example 5: Phase 3 SLE Study Design

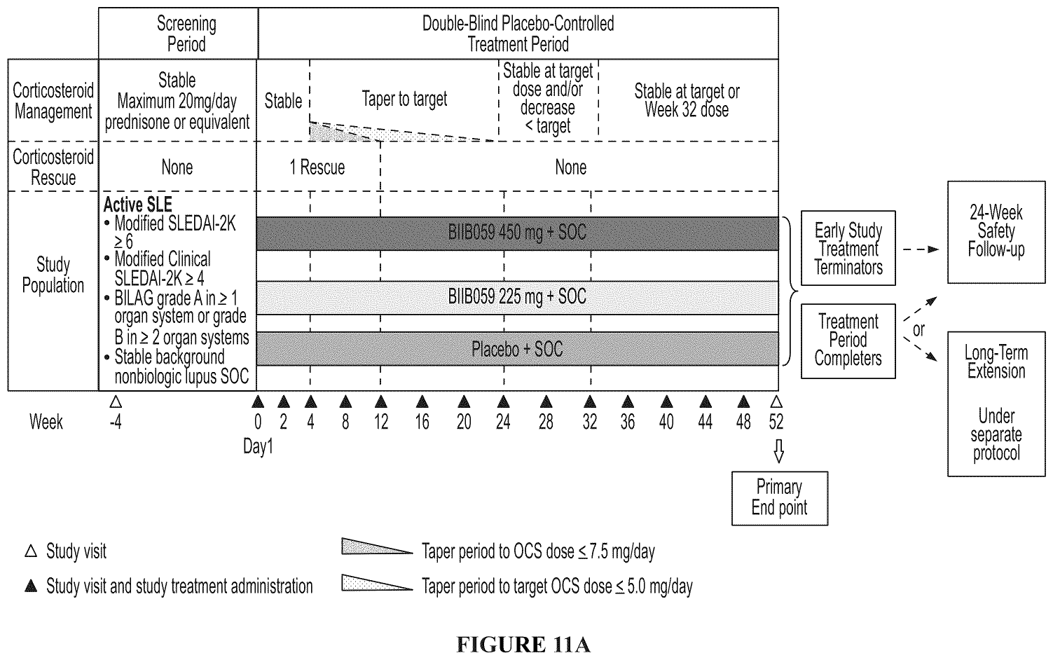

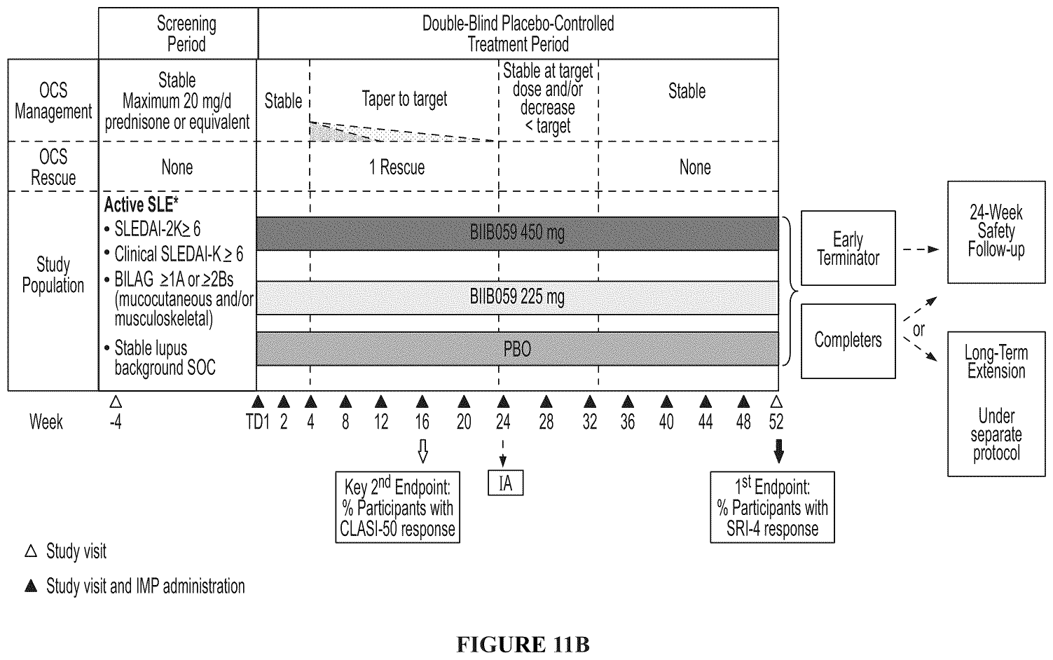

The Phase 3 study will be a randomized, double-blind, placebo controlled, multicenter study to evaluate the efficacy and safety of BIIB059 in participants with active SLE (FIG. 11A). Approximately 540 participants (180 per treatment arm, per study) will be enrolled globally and randomized in a 1:1:1 ratio to receive BIIB059 450 mg or BIIB059 225 mg Q4W SC or matching placebo, respectively, for a 52-week DBPC treatment period followed by a SFU period (off-treatment) of 24 weeks. Participants who complete the Phase 3 studies will be eligible to participate in a separate long-term extension (LTE) study.

All participants will have SLE diagnosis and moderate to severe disease activity. All participants will be treated with stable lupus background non-biologic standard of care (SoC), with a requirement for a therapy initiation >=12 weeks prior to study entry, and a stable dose for at least 4 weeks prior to randomization. Four weeks after the first dose, initiation of a mandatory corticosteroid taper, to achieve a corticosteroid target dose of 7.5 mg/day, will be required for participants who are treated with a dose >10 mg/day at baseline. One corticosteroid rescue will be allowed during Week 5 and Week 12.

The study populations will include participants with active SLE, who are treated with background lupus SoC.
1. At the time of informed consent, the participant must be ≥16 years of age, where allowed by local regulations.
2. Participant must be diagnosed with SLE and must meet the 2019 EULAR/ACR classification criteria for SLE., at least 24 weeks before Screening by a qualified physician, as classified by
3. Participant has a modified SLEDAI-2K≥6 (excluding alopecia, lupus-related headache and organic brain syndrome) at Screening (adjudicated) and randomization as follow:
   a. If 4 points of the required SLEDAI-2K entry points are for arthritis, there must also be at least 4 joints that are both swollen and tender (with at least 4 occurring in the PIP, MCP, or wrist joints), based on the 28-joint count assessment. If 2 points of the required SLEDAI-2K entry points are due to rash, the rash must be attributable to ACLE, SCLE, and/or CCLE (e.g., DLE) skin manifestation
4. Participant has a modified clinical SLEDAI-2K≥4 (excluding anti-ds DNA, low complement C3 and/or C4, alopecia, fever, lupus-related headache, and organic brain syndrome) at Screening (adjudicated) and randomization.
5. Participant has BILAG-2004 grade A in ≥1 organ system or BILAG-2004 grade B in ≥2 organ systems, at Screening (adjudicated) and randomization.

Participant must be treated with ONE of the following stable background lupus SOC lupus SOC therapies, initiated ≥12 weeks prior to Screening and at stable dose ≥4 weeks prior to randomization
a. Antimalarial treatment in combination with corticosteroids and/or immunosuppressants.
b. Antimalarial as stand-alone treatment
c. Treatment with oral corticosteroid (OCS) and/or immunosuppressants.
6. Participant has ONE of the following, per the central laboratory at Screening:
   a. ANA ≥1:80 by immunofluorescent assay
   b. Anti-dsDNA antibodies above normal levels
   c. Anti-Smith antibody above normal levels

Primary and key secondary objectives and endpoints of the study are presented in Table 3.

TABLE 3
Primary and Key Secondary Objectives and Endpoints

| Primary Objective | Primary Endpoints |
|---|---|
| To demonstrate efficacy of BIIB059 | Proportion of participants who achieved an SRI- |
| compared with placebo in participants with | 4 response at Week 52. |
| active SLE, who are receiving background | The composite endpoint SRI-4 is defined by |
| lupus SOC therapy in reducing disease | the following criteria: |
| activity | i. A reduction from Baseline of ≥4 points in |
| | SLEDAI-2K score; and |
| | ii. No new organ system affected, as defined by no new |
| | organ system with BILAG-2004 grade A and no more |
| | than 1 new organ system with BILAG-2004 grade B |
| | compared with Baseline; and |
| | iii. No worsening from Baseline in lupus disease |
| | activity as defined by <0.3-point increase on 3-point |
| | PGA-VAS; and |
| | iv. No changes to protocol-specified medication rules. |
| Key Secondary Objectives | Key Secondary Endpoints |
| To demonstrate early onset of efficacy of | Proportion of participants who achieved an SRI- |
| BIIB059 compared with placebo in | 4 response at Week 24. |
| participants with active SLE, and who are | |
| receiving background lupus SOC therapy in | |
| reducing disease activity | |
| To demonstrate organ-specific efficacy of | Proportion of participants with at least 4 joints |
| BIIB059 compared with placebo in | (both swollen and tender) at Baseline who |
| participants with active SLE, who are | achieved a Joint-50 |
| receiving background lupus SOC therapy in | response at Week 52. |
| reducing joint disease activity | |
| To demonstrate effect of BIIB059 compared | Proportion of participants with OCS ≥10 |
| with placebo in reducing OCS use | mg/day at Baseline who have OCS |
| | reduction to ≤7.5 mg/day at Week 40, |
| | which is sustained through Week 52 with |
| | no disease worsening from Week 40 to |
| | Week 52. |
| To demonstrate organ-specific efficacy of | Proportion of participants with a CLASI-A |
| BIIB059 compared with placebo in | score ≥10 at Baseline who achieved a |
| participants with active SLE, who are | CLASI-50 response at Week 16. |
| receiving background lupus SOC therapy in | |
| reducing skin disease activity | |
| To demonstrate efficacy of BIIB059 | Annualized flare rate through Week 52. |
| compared with placebo in participants with | |
| active SLE, who are receiving background | |
| lupus SOC therapy in reducing occurrence of | |
| flare up to Week 52 | |

An alternate schematic diagram of a SLE study design is depicted in FIG. 11B.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating cutaneous lupus erythematosus (CLE) or systemic lupus erythematosus (SLE) in a human subject in need thereof, the method comprising administering subcutaneously to the human subject an anti-BDCA2 antibody at a dose of 225 mg every four weeks, wherein the anti-BDCA2 antibody comprises an immunoglobulin heavy chain variable domain (VH) and an immunoglobulin light chain variable domain (VL), the VH and VL, respectively, comprising:
(a) VH complementarity determining regions (CDRs) VH-CDR1, VH-CDR2, and VH-CDR3, wherein
VH-CDR1 consists of the amino acid sequence set forth in SEQ ID NO:1;
VH-CDR2 consists of the amino acid sequence set forth in SEQ ID NO:2; and
VH-CDR3 consists of the amino acid sequence set forth in SEQ ID NO:3; and
(b) VL CDRs VL-CDR1, VL-CDR2, and VL-CDR3, wherein
VL-CDR1 consists of the amino acid sequence set forth in SEQ ID NO:4;
VL-CDR2 consists of the amino acid sequence set forth in SEQ ID NO:5; and
VL-CDR3 consists of the amino acid sequence set forth in SEQ ID NO:6.

2. The method of claim 1, wherein the human subject is administered a loading dose of the anti-BDCA2 antibody two weeks after the first administration of the anti-BDCA2 antibody.

3. A method of treating cutaneous lupus erythematosus (CLE) or systemic lupus erythematosus (SLE) in a human subject in need thereof, the method comprising administering subcutaneously to the human subject an anti-BDCA2 antibody at a dose of 225 mg every four weeks, wherein the human subject is administered a loading dose of 225 mg of the anti-BDCA2 antibody two weeks after the first administration of the anti-BDCA2 antibody, wherein the anti-BDCA2 antibody comprises an immunoglobulin heavy chain variable domain (VH) and an immunoglobulin light chain variable domain (VL), the VH and VL, respectively, comprising:
(a) VH complementarity determining regions (CDRs) VH-CDR1, VH-CDR2, and VH-CDR3, wherein
VH-CDR1 consists of the amino acid sequence set forth in SEQ ID NO:1;
VH-CDR2 consists of the amino acid sequence set forth in SEQ ID NO:2; and
VH-CDR3 consists of the amino acid sequence set forth in SEQ ID NO:3; and
(b) VL CDRs VL-CDR1, VL-CDR2, and VL-CDR3, wherein
VL-CDR1 consists of the amino acid sequence set forth in SEQ ID NO:4;
VL-CDR2 consists of the amino acid sequence set forth in SEQ ID NO:5; and
VL-CDR3 consists of the amino acid sequence set forth in SEQ ID NO:6.

4. The method of any one of claims 1 to 3, wherein the human subject is administered a second loading dose of the anti-BDCA2 antibody.

5. The method of claim 4, wherein the second loading dose is 225 mg.

6. The method of any one of claims 1 to 3, wherein the anti-BDCA2 antibody is administered at a dose of 225 mg every four weeks over at least 16 weeks.

7. The method of any one of claims 1 to 6, wherein the anti-BDCA2 antibody is administered at a dose of 225 mg every four weeks over at least 52 weeks.

8. The method of any one of claims 1 to 5, wherein at least four doses of the anti-BDCA2 antibody are administered to the human subject.

9. The method of any one of claims 1 to 5, wherein at least twelve doses of the anti-BDCA2 antibody are administered to the human subject.

10. The method of any one of claims 1 to 5, wherein at least fourteen doses of the anti-BDCA2 antibody are administered to the human subject.

11. The method of any one of claims 1 to 5, wherein at least sixteen doses of the anti-BDCA2 antibody are administered to the human subject.

12. The method of any one of claims 1 to 11, wherein the CLE disease is moderate CLE.

13. The method of any one of claims 1 to 11, wherein the CLE disease is severe CLE.

14. The method of any one of claims 1 to 11, wherein the CLE type is acute CLE (ACLE).

15. The method of any one of claims 1 to 11, wherein the CLE type is subacute CLE (SCLE).

16. The method of any one of claims 1 to 11, wherein the CLE type is chronic CLE (CCLE).

17. The method of claim 16, wherein the CCLE is discoid lupus erythematosus (DLE).

18. The method of any one of claims 1 to 11, wherein the CLE disease is active CLE.

19. The method of claim 18, wherein the active CLE is with systemic manifestations of lupus and the human subject is intolerant or refractory to antimalarial therapy.

20. The method of claim 18, wherein the active CLE is without systemic manifestations of lupus and the human subject is intolerant or refractory to antimalarial therapy.

21. The method of any one of claims 1 to 20, wherein the human subject achieves clinically meaningful reduction in Cutaneous Lupus Erythematosus Disease Area and Severity Index-A (CLASI-A) score from baseline about sixteen weeks to about 24 weeks after the first administration of the anti-BDCA2 antibody.

22. The method of any one of claims 1 to 20, wherein the human subject achieves a clinically meaningful reduction from baseline in disease activity on an CLA-IGA-R specific for CLE about sixteen weeks to about 24 weeks after the first administration of the anti-BDCA2 antibody.

23. The method of any one of claims 1 to 11, wherein the SLE disease is active SLE.

24. The method of any one of claims 1 to 11, wherein the human subject has active, autoantibody-positive SLE.

25. The method of any one of claims 1 to 11, wherein the human subject has active, autoantibody-positive SLE and the human subject is receiving the standard of care therapy for SLE.

26. The method of any one of claims 1 to 11, wherein the SLE disease is moderate SLE.

27. The method of any one of claims 1 to 11, wherein the SLE disease is severe SLE.

28. The method of any one of claims 1 to 11, wherein the human subject has a modified SLEDAI-2K≥6 excluding alopecia, fever, lupus-related headache, and organic brain syndrome at initiation of treatment.

29. The method of any one of claims 1 to 11, wherein the human subject has a clinical SLEDAI-2K≥4 excluding alopecia, lupus-related headache and organic brain disease, anti-ds DNA, low complement C3 and/or C4, or fever, at initiation of treatment.

30. The method of any one of claims 1 to 11, wherein the human subject has BILAG-2004 grade A in ≥1 organ system or BILAG-2004 grade B in ≥2 organ systems at initiation of treatment.

31. The method of any one of claims 1 to 11, wherein the human subject is treated with an antimalarial, an oral corticosteroid, and/or an immunosuppressant prior to initiation of treatment with the anti-BDCA2 antibody.

32. The method of any one of claims 1 to 31, wherein the anti-BDCA2 antibody is formulated as a sterile, liquid pharmaceutical composition comprising:
the anti-BDCA2 antibody at a concentration of 150 mg/ml;
sucrose at an a concentration of 3%;
L-histidine at a concentration of 20 mM;
L-Arginine HCl at a concentration of 100 mM;
glutathione at a concentration of 0.4 mM; and
polysorbate 80 (PS80) at a concentration of 0.05%,
wherein the pharmaceutical composition has a pH of 5.7.

33. The method of any one of claims 1 to 32, wherein:
(i) the VH comprises a sequence at least 80% identical to the amino acid sequence of SEQ ID NO:7 and the VL comprises a sequence at least 80% identical to the amino acid sequence of SEQ ID NO:8;
(ii) the VH comprises a sequence at least 90% identical to the amino acid sequence of SEQ ID NO:7 and the VL comprises a sequence at least 90% identical to the amino acid sequence of SEQ ID NO:8; or
(iii) the VH comprises the amino acid sequence set forth in SEQ ID NO:7 and the VL comprises the amino acid sequence set forth in SEQ ID NO:8.

34. The method of any one of claims 1 to 33, wherein the anti-BDCA2 antibody comprises an immunoglobulin heavy chain and an immunoglobulin light chain, wherein:
(i) the heavy chain comprises a sequence at least 80% identical to the amino acid sequence of SEQ ID NO:9 and the light chain comprises a sequence at least 80% identical to the amino acid sequence of SEQ ID NO:10;
(ii) the heavy chain comprises a sequence at least 90% identical to the amino acid sequence of SEQ ID NO:9 and the light chain comprises a sequence at least 90% identical to the amino acid sequence of SEQ ID NO:10; or
(iii) the heavy chain comprises the amino acid sequence set forth in SEQ ID NO:9 and the light chain comprises the amino acid sequence set forth in SEQ ID NO:10.

35. A pre-filled syringe comprising a sterile preparation of an anti-BDCA2 antibody, wherein the pre-filled syringe is adapted for subcutaneous administration of the anti-BDCA2 antibody at a fixed dose of 225 mg, and wherein the anti-BDCA2 antibody comprises an immunoglobulin heavy chain variable domain (VH) and an immunoglobulin light chain variable domain (VL), the VH and VL, respectively, comprising:
(a) VH complementarity determining regions (CDRs) VH-CDR1, VH-CDR2, and VH-CDR3, wherein
VH-CDR1 comprises the amino acid sequence set forth in SEQ ID NO:1;
VH-CDR2 comprises the amino acid sequence set forth in SEQ ID NO:2; and
VH-CDR3 comprises the amino acid sequence set forth in SEQ ID NO:3; and
(b) VL CDRs VL-CDR1, VL-CDR2, and VL-CDR3, wherein
VL-CDR1 comprises the amino acid sequence set forth in SEQ ID NO:4;
VL-CDR2 comprises the amino acid sequence set forth in SEQ ID NO:5; and
VL-CDR3 comprises the amino acid sequence set forth in SEQ ID NO:6.

36. The pre-filled syringe of claim 35, wherein:
(i) the VH comprises a sequence at least 80% identical to the amino acid sequence of SEQ ID NO:7 and the VL comprises a sequence at least 80% identical to the amino acid sequence of SEQ ID NO:8;
(ii) the VH comprises a sequence at least 90% identical to the amino acid sequence of SEQ ID NO:7 and the VL comprises a sequence at least 90% identical to the amino acid sequence of SEQ ID NO:8; or
(iii) the VH comprises the amino acid sequence set forth in SEQ ID NO:7 and the VL comprises the amino acid sequence set forth in SEQ ID NO:8.

37. The pre-filled syringe of claim 35 or 36, wherein the anti-BDCA2 antibody comprises an immunoglobulin heavy chain and an immunoglobulin light chain, wherein:
(i) the heavy chain comprises a sequence at least 80% identical to the amino acid sequence of SEQ ID NO:9 and the light chain comprises a sequence at least 80% identical to the amino acid sequence of SEQ ID NO:10;
(ii) the heavy chain comprises a sequence at least 90% identical to the amino acid sequence of SEQ ID NO:9 and the light chain comprises a sequence at least 90% identical to the amino acid sequence of SEQ ID NO:10; or
(iii) the heavy chain comprises the amino acid sequence set forth in SEQ ID NO:9 and the light chain comprises the amino acid sequence set forth in SEQ ID NO:10.

38. The pre-filled syringe of any one of claims 35 to 37, wherein the anti-BDCA2 antibody is formulated as a sterile, liquid pharmaceutical composition comprising:
the anti-BDCA2 antibody at a concentration of 150 mg/ml;
sucrose at an a concentration of 3%;
L-histidine at a concentration of 20 mM;
L-Arginine HCl at a concentration of 100 mM;
glutathione at a concentration of 0.4 mM; and
polysorbate 80 (PS80) at a concentration of 0.05%,
wherein the pharmaceutical composition has a pH of 5.7.

39. The pre-filled syringe of any one of claims 35 to 38, wherein the pre-filled syringe is a United States Pharmacopeia or European Pharmacopeia, Type 1, clear glass vial that is stoppered with a rubber stopper.
